SEQUENCE LISTING (342 sequences)

SEQ ID NO: 1 - DNA, 2211 bp, synthetic construct
  source 1..2211: mol_type = other DNA; note = AAV-LK03-R594E+ D598H -VP1
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactgaaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 2 - protein, 736 aa, synthetic construct
  source 1..736: note = AAV-LK03-R594E+ D598H -VP1
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTETVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 2208 bp, synthetic construct
  source 1..2208: mol_type = other DNA; note = AAVC11.11
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgtcccctgtaa

SEQ ID NO: 4 - protein, 735 aa, synthetic construct
  source 1..735: note = AAVC11.11
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 735 aa, Adeno-associated dependoparvovirus A [sequence omitted]

SEQ ID NO: 6 - protein, 34 aa, Adeno-associated dependoparvovirus A
  source 1..34: note = AAV1 capsid positions 248-281
ALPTYNNHLYKQISSASTGASNDNHYFGYSTPWG

SEQ ID NO: 7 - protein, 33 aa, Adeno-associated dependoparvovirus A
  source 1..33: note = AAV2/3b capsid positions 248-280
ALPTYNNHLYKQISSQSGASNDNHYFGYSTPWG

SEQ ID NO: 8 - protein, 34 aa, Adeno-associated dependoparvovirus A
  source 1..34: note = AAV7 capsid positions 249-282
ALPTYNNHLYKQISSETAGSTNDNTYFGYSTPWG

SEQ ID NO: 9 - protein, 35 aa, Adeno-associated dependoparvovirus A
  source 1..35: note = AAV8 capsid positions 249-283
ALPTYNNHLYKQISSGTSGGATNDNTYFGYSTPWG

SEQ ID NO: 10 - protein, 35 aa, Adeno-associated dependoparvovirus A
  source 1..35: note = AAV9 capsid positions 248-282
ALPTYNNHLYKQISNSTSGGSSNDNAYFGYSTPWG

SEQ ID NO: 11 - protein, 35 aa, Adeno-associated dependoparvovirus A
  source 1..35: note = AAV10 capsid positions 249-283
ALPTYNNHLYKQISNGTSGGSTNDNTYFGYSTPWG

SEQ ID NO: 12 - protein, 10 aa, Adeno-associated dependoparvovirus A
  source 1..10: note = AAV2 capsid positions 262-271
SQSGASNDNH

SEQ ID NO: 13 - DNA, 56 bp, synthetic construct
  source 1..56: mol_type = other DNA; note = Fwd 10aa
catctctacaagcaaatctccarcvvwwcwggakctwcwaacgacaacvmctactt

SEQ ID NO: 14 - DNA, 59 bp, synthetic construct
  source 1..59: mol_type = other DNA; note = Fwd 11aa
catctctacaagcaaatctccarcvvwwcwrvcggakctwcwaacgacaacvmctactt

SEQ ID NO: 15 - DNA, 62 bp, synthetic construct
  source 1..62: mol_type = other DNA; note = Fwd 12aa
catctctacaagcaaatctccarcvvwwcwrvcggaggakctwcwaacgacaacvmctactt

SEQ ID NO: 16 - DNA, 53 bp, synthetic construct
  source 1..53: mol_type = other DNA; note = Rev common 1
ggagatttgcttgtagagatggttgttgtaagtgggcagggcccaggttctgg

SEQ ID NO: 17 - DNA, 53 bp, synthetic construct
  source 1..53: mol_type = other DNA; note = Rev common 2
ggagatttgcttgtagagatgattattgtacgtgggcagggcccaggttctgg

SEQ ID NO: 18 - DNA, 53 bp, synthetic construct
  source 1..53: mol_type = other DNA; note = Rev common 3
ggagatttgcttgtagagatggttattataggtgggcagggcccaggttctgg

SEQ ID NO: 19 - DNA, 21 bp, synthetic construct
  source 1..21: mol_type = other DNA; note = Forward NGS
accaccagcaccagaacctgg

SEQ ID NO: 20 - DNA, 32 bp, synthetic construct
  source 1..32: mol_type = other DNA; note = Reverse NGS
gcagtggaatctgttaaagtcaaaataccccc

SEQ ID NO: 21 - protein, 12 aa, synthetic construct
SRSAGGASNDNA

SEQ ID NO: 22 - protein, 12 aa, synthetic construct
SRSSGGSTNDNA

SEQ ID NO: 23 - protein, 12 aa, synthetic construct
NRTGGGATNDNA

SEQ ID NO: 24 - protein, 11 aa, synthetic construct
NTTAGSSNDNP

SEQ ID NO: 25 - protein, 12 aa, synthetic construct
NRTAGGSTNDNH

SEQ ID NO: 26 - protein, 12 aa, synthetic construct
SRSSGGSSNDNT

SEQ ID NO: 27 - protein, 11 aa, synthetic construct
SKSTGASNDNN

SEQ ID NO: 28 - protein, 12 aa, synthetic construct
SRSSGGASNDNH

SEQ ID NO: 29 - protein, 12 aa, synthetic construct
NKTGGGASNDNT

SEQ ID NO: 30 - protein, 12 aa, synthetic construct
NKTNGGATNDNH

SEQ ID NO: 31 - protein, 12 aa, synthetic construct
NRTTGGATNDNH

SEQ ID NO: 32 - protein, 11 aa, synthetic construct
NKTGGASNDNT

SEQ ID NO: 33 - protein, 12 aa, synthetic construct
SKSAGGSSNDNA

SEQ ID NO: 34 - protein, 12 aa, synthetic construct
NRTSGGASNDNH

SEQ ID NO: 35 - protein, 12 aa, synthetic construct
SRSTGGASNDNA

SEQ ID NO: 36 - protein, 11 aa, synthetic construct
SKSTGASNDNA

SEQ ID NO: 37 - protein, 12 aa, synthetic construct
NKTAGGATNDNT

SEQ ID NO: 38 - protein, 12 aa, synthetic construct
NRSSGGATNDNA

SEQ ID NO: 39 - protein, 12 aa, synthetic construct
SRSSGGASNDNA

SEQ ID NO: 40 - protein, 12 aa, synthetic construct
SKTTGGSSNDNA

SEQ ID NO: 41 - protein, 12 aa, synthetic construct
SKSAGGASNDNH

SEQ ID NO: 42 - protein, 12 aa, synthetic construct
SRSTGGASNDNN

SEQ ID NO: 43 - protein, 12 aa, synthetic construct
SRSTGGSSNDNA

SEQ ID NO: 44 - protein, 12 aa, synthetic construct
NKTAGGSTNDNH

SEQ ID NO: 45 - protein, 12 aa, synthetic construct
NRTNGGATNDNT

SEQ ID NO: 46 - protein, 12 aa, synthetic construct
NRTTGGSTNDNN

SEQ ID NO: 47 - protein, 12 aa, synthetic construct
NKTTGGATNDNH

SEQ ID NO: 48 - protein, 10 aa, synthetic construct
NNSGATNDNH

SEQ ID NO: 49 - protein, 11 aa, synthetic construct
NHSNGATNDNN

SEQ ID NO: 50 - protein, 11 aa, synthetic construct
SKTSGSSNDNH

SEQ ID NO: 51 - protein, 12 aa, synthetic construct
SRSAGGSSNDNT

SEQ ID NO: 52 - protein, 11 aa, synthetic construct
NGTAGSSNDNN

SEQ ID NO: 53 - protein, 12 aa, synthetic construct
NRTSGGASNDNA

SEQ ID NO: 54 - protein, 12 aa, synthetic construct
SSSSGGASNDNN

SEQ ID NO: 55 - protein, 12 aa, synthetic construct
SRSSGGSSNDNA

SEQ ID NO: 56 - protein, 12 aa, synthetic construct
NKTNGGASNDNN

SEQ ID NO: 57 - protein, 12 aa, synthetic construct
NKTGGGASNDNH

SEQ ID NO: 58 - protein, 12 aa, synthetic construct
SRTTGGSSNDNH

SEQ ID NO: 59 - protein, 12 aa, synthetic construct
SRTAGGATNDNN

SEQ ID NO: 60 - protein, 12 aa, synthetic construct
SRSGGGSSNDNT

SEQ ID NO: 61 - protein, 11 aa, synthetic construct
NHTSGSSNDNH

SEQ ID NO: 62 - protein, 11 aa, synthetic construct
SKSTGSTNDNH

SEQ ID NO: 63 - protein, 11 aa, synthetic construct
NSTGGSSNDNA

SEQ ID NO: 64 - protein, 12 aa, synthetic construct
NESGGGASNDNN

SEQ ID NO: 65 - protein, 11 aa, synthetic construct
NNSSGATNDNN

SEQ ID NO: 66 - protein, 12 aa, synthetic construct
NRTNGGATNDNH

SEQ ID NO: 67 - protein, 12 aa, synthetic construct
NRTGGGATNDNH

SEQ ID NO: 68 - protein, 12 aa, synthetic construct
NASTGGATNDNN

SEQ ID NO: 69 - protein, 11 aa, synthetic construct
SDTNGATNDNN

SEQ ID NO: 70 - protein, 10 aa, synthetic construct
SASGSSNDNH

SEQ ID NO: 71 - protein, 12 aa, synthetic construct
NDSTGGATNDNN

SEQ ID NO: 72 - protein, 12 aa, synthetic construct
NATTGGASNDNA

SEQ ID NO: 73 - protein, 11 aa, synthetic construct
SKTNGSSNDNH

SEQ ID NO: 74 - protein, 11 aa, synthetic construct
STTSGASNDNA

SEQ ID NO: 75 - protein, 12 aa, synthetic construct
SHTAGGSSNDNH

SEQ ID NO: 76 - protein, 11 aa, synthetic construct
STSAGASNDNH

SEQ ID NO: 77 - protein, 11 aa, synthetic construct
NRSGGATNDNH

SEQ ID NO: 78 - protein, 11 aa, synthetic construct
NTSGGSSNDNN

SEQ ID NO: 79 - protein, 12 aa, synthetic construct
NHSAGGASNDNA

SEQ ID NO: 80 - protein, 12 aa, synthetic construct
SETSGGSSNDNH

SEQ ID NO: 81 - protein, 12 aa, synthetic construct
SASNGGSSNDNA

SEQ ID NO: 82 - protein, 12 aa, synthetic construct
SDTGGGASNDNT

SEQ ID NO: 83 - protein, 12 aa, synthetic construct
SSSGGGATNDNA

SEQ ID NO: 84 - protein, 12 aa, synthetic construct
SNSAGGSSNDNA

SEQ ID NO: 85 - protein, 12 aa, synthetic construct
SSTNGGSTNDNH

SEQ ID NO: 86 - protein, 12 aa, synthetic construct
SGTGGGASNDNA

SEQ ID NO: 87 - protein, 12 aa, synthetic construct
SGTTGGATNDNN

SEQ ID NO: 88 - protein, 12 aa, synthetic construct
SGTSGGASNDNA

SEQ ID NO: 89 - protein, 11 aa, synthetic construct
NDSSGSSNDNT

SEQ ID NO: 90 - protein, 11 aa, synthetic construct
NKSTGASNDNN

SEQ ID NO: 91 - protein, 11 aa, synthetic construct
NQTGGSSNDNA

SEQ ID NO: 92 - protein, 10 aa, synthetic construct
SNSGATNDNH

SEQ ID NO: 93 - protein, 12 aa, synthetic construct
SHSSGGSSNDNH

SEQ ID NO: 94 - protein, 12 aa, synthetic construct
SQTGGGASNDNH

SEQ ID NO: 95 - protein, 11 aa, synthetic construct
SKTSGSSNDNN

SEQ ID NO: 96 - protein, 12 aa, synthetic construct
SRSSGGASNDNT

SEQ ID NO: 97 - protein, 11 aa, synthetic construct
NRSTGASNDNN

SEQ ID NO: 98 - protein, 12 aa, synthetic construct
NGSNGGASNDNN

SEQ ID NO: 99 - protein, 12 aa, synthetic construct
SRTNGGSTNDNA

SEQ ID NO: 100 - protein, 11 aa, synthetic construct
SSSGGSTNDNA

SEQ ID NO: 101 - protein, 12 aa, synthetic construct
SGTNGGSTNDNT

SEQ ID NO: 102 - protein, 12 aa, synthetic construct
SRSDGGSTNDNN

SEQ ID NO: 103 - protein, 12 aa, synthetic construct
SGSNGGASNDNN

SEQ ID NO: 104 - protein, 12 aa, synthetic construct
NRSNGGSSNDNH

SEQ ID NO: 105 - protein, 12 aa, synthetic construct
SKTNGGASNDNH

SEQ ID NO: 106 - protein, 12 aa, synthetic construct
SRSSGGATNDNA

SEQ ID NO: 107 - protein, 12 aa, synthetic construct
SSSTGGATNDNA

SEQ ID NO: 108 - protein, 12 aa, synthetic construct
NDTGGGATNDNP

SEQ ID NO: 109 - protein, 12 aa, synthetic construct
SGSGGGSTNDNA

SEQ ID NO: 110 - protein, 12 aa, synthetic construct
SSSGGGSTNDNA

SEQ ID NO: 111 - protein, 11 aa, synthetic construct
SDTSGATNDNA

SEQ ID NO: 112 - protein, 12 aa, synthetic construct
NASTGGATNDNP

SEQ ID NO: 113 - protein, 12 aa, synthetic construct
NKTSGGSSNDNA

SEQ ID NO: 114 - protein, 12 aa, synthetic construct
NATGGGATNDNP

SEQ ID NO: 115 - protein, 12 aa, synthetic construct
SKTNGGSTNDNA

SEQ ID NO: 116 - protein, 12 aa, synthetic construct
SGTNGGSSNDNT

SEQ ID NO: 117 - protein, 12 aa, synthetic construct
NTTAGGSTNDNN

SEQ ID NO: 118 - protein, 12 aa, synthetic construct
SQTGGGASNDNT

SEQ ID NO: 119 - protein, 11 aa, synthetic construct
NTTTGATNDNT

SEQ ID NO: 120 - protein, 11 aa, synthetic construct
SHTAGSSNDNA

SEQ ID NO: 121 - protein, 11 aa, synthetic construct
SKSSGSSNDNA

SEQ ID NO: 122 - protein, 12 aa, synthetic construct
SSSDGGSTNDNA

SEQ ID NO: 123 - protein, 12 aa, synthetic construct
SNTTGGSTNDNH

SEQ ID NO: 124 - protein, 12 aa, synthetic construct
NQTGGGATNDNT

SEQ ID NO: 125 - protein, 12 aa, synthetic construct
NRSDGGSTNDNN

SEQ ID NO: 126 - protein, 12 aa, synthetic construct
SGTGGGASNDNH

SEQ ID NO: 127 - protein, 12 aa, synthetic construct
SNSSGGSTNDNA

SEQ ID NO: 128 - protein, 12 aa, synthetic construct
NSTNGGATNDNA

SEQ ID NO: 129 - protein, 12 aa, synthetic construct
NSSSGGATNDNT

SEQ ID NO: 130 - protein, 12 aa, synthetic construct
SQSSGGATNDNH

SEQ ID NO: 131 - protein, 12 aa, synthetic construct
SKSDGGSTNDNH

SEQ ID NO: 132 - protein, 12 aa, synthetic construct
SNTAGGSSNDNT

SEQ ID NO: 133 - protein, 12 aa, synthetic construct
NESAGGSSNDNN

SEQ ID NO: 134 - protein, 12 aa, synthetic construct
NHSGGGATNDNP

SEQ ID NO: 135 - protein, 12 aa, synthetic construct
SSSSGGATNDNA

SEQ ID NO: 136 - protein, 11 aa, synthetic construct
SHTDGSTNDNH

SEQ ID NO: 137 - protein, 12 aa, synthetic construct
SATDGGSTNDNA

SEQ ID NO: 138 - protein, 11 aa, synthetic construct
SKSSGATNDNT

SEQ ID NO: 139 - protein, 12 aa, synthetic construct
NRSTGGSTNDNT

SEQ ID NO: 140 - protein, 12 aa, synthetic construct
SPTDGGSTNDNN

SEQ ID NO: 141 - protein, 12 aa, synthetic construct
SESAGGSTNDNN

SEQ ID NO: 142 - protein, 12 aa, synthetic construct
NSSGGGSTNDNT

SEQ ID NO: 143 - protein, 12 aa, synthetic construct
NDTSGGATNDNT

SEQ ID NO: 144 - protein, 10 aa, synthetic construct
NNTGSSNDNH

SEQ ID NO: 145 - protein, 12 aa, synthetic construct
NKSSGGSTNDNN

SEQ ID NO: 146 - protein, 12 aa, synthetic construct
STTSGGSTNDND

SEQ ID NO: 147 - protein, 12 aa, synthetic construct
SHSAGGSTNDNA

SEQ ID NO: 148 - protein, 12 aa, synthetic construct
SDTAGGSTNDNN

SEQ ID NO: 149 - protein, 11 aa, synthetic construct
SRTDGSTNDNP

SEQ ID NO: 150 - protein, 10 aa, synthetic construct
NHSGATNDNP

SEQ ID NO: 151 - protein, 11 aa, synthetic construct
NSSGGATNDNA

SEQ ID NO: 152 - protein, 11 aa, synthetic construct
NNSNGSSNDNA

SEQ ID NO: 153 - protein, 12 aa, synthetic construct
NPSNGGSSNDND

SEQ ID NO: 154 - protein, 12 aa, synthetic construct
SQTNGGSTNDND

SEQ ID NO: 155 - protein, 10 aa, synthetic construct
SDSGSSNDNT

SEQ ID NO: 156 - protein, 11 aa, synthetic construct
NESAGATNDNN

SEQ ID NO: 157 - protein, 12 aa, synthetic construct
NPSAGGSSNDNN

SEQ ID NO: 158 - protein, 10 aa, synthetic construct
SSTGATNDNT

SEQ ID NO: 159 - protein, 12 aa, synthetic construct
SESNGGSSNDNT

SEQ ID NO: 160 - protein, 11 aa, synthetic construct
SDTNGSTNDNA

SEQ ID NO: 161 - protein, 10 aa, synthetic construct
NPSGASNDNT

SEQ ID NO: 162 - protein, 12 aa, synthetic construct
STSGGGSTNDNP

SEQ ID NO: 163 - protein, 12 aa, synthetic construct
NHSTGGATNDND

SEQ ID NO: 164 - protein, 11 aa, synthetic construct
NDSGGATNDNT

SEQ ID NO: 165 - protein, 12 aa, synthetic construct
SHTDGGSSNDNA

SEQ ID NO: 166 - protein, 11 aa, synthetic construct
SKSSGATNDNA

SEQ ID NO: 167 - protein, 10 aa, synthetic construct
SKTGSTNDND

SEQ ID NO: 168 - protein, 12 aa, synthetic construct
SQTGGGSSNDNP

SEQ ID NO: 169 - protein, 11 aa, synthetic construct
SHTSGSSNDNP

SEQ ID NO: 170 - protein, 11 aa, synthetic construct
NASSGSSNDND

SEQ ID NO: 171 - protein, 12 aa, synthetic construct
SSTAGGSTNDNH

SEQ ID NO: 172 - protein, 12 aa, synthetic construct
NGSDGGSTNDND

SEQ ID NO: 173 - protein, 12 aa, synthetic construct
STSSGGSSNDNP

SEQ ID NO: 174 - protein, 11 aa, synthetic construct
SNTGGASNDNA

SEQ ID NO: 175 - protein, 11 aa, synthetic construct
NDTDGASNDNA

SEQ ID NO: 176 - protein, 11 aa, synthetic construct
SDTTGATNDNN

SEQ ID NO: 177 - protein, 10 aa, synthetic construct
SPSGSTNDNN

SEQ ID NO: 178 - protein, 11 aa, synthetic construct
NRSAGATNDNP

SEQ ID NO: 179 - protein, 10 aa, synthetic construct
NTSGATNDNT

SEQ ID NO: 180 - protein, 11 aa, synthetic construct
NRTGGSTNDNA

SEQ ID NO: 181 - protein, 11 aa, synthetic construct
SETGGATNDNT

SEQ ID NO: 182 - protein, 12 aa, synthetic construct
SATNGGSSNDNA

SEQ ID NO: 183 - protein, 11 aa, synthetic construct
SKTDGSSNDNT

SEQ ID NO: 184 - protein, 11 aa, synthetic construct
NGTSGSTNDNN

SEQ ID NO: 185 - protein, 12 aa, synthetic construct
NNSSGGSSNDNA

SEQ ID NO: 186 - protein, 12 aa, synthetic construct
NQSTGGSSNDNT

SEQ ID NO: 187 - protein, 11 aa, synthetic construct
NQSNGSTNDNA

SEQ ID NO: 188 - protein, 11 aa, synthetic construct
SSTTGSTNDNA

SEQ ID NO: 189 - protein, 11 aa, synthetic construct
NDSGGSTNDNT

SEQ ID NO: 190 - protein, 12 aa, synthetic construct
NETSGGSSNDNA

SEQ ID NO: 191 - protein, 11 aa, synthetic construct
NSTDGATNDNP

SEQ ID NO: 192 - protein, 12 aa, synthetic construct
NRSSGGSTNDNH

SEQ ID NO: 193 - protein, 11 aa, synthetic construct
SKTGGATNDNA

SEQ ID NO: 194 - protein, 12 aa, synthetic construct
NKSTGGATNDNT

SEQ ID NO: 195 - protein, 11 aa, synthetic construct
NSSTGSSNDNA

SEQ ID NO: 196 - protein, 12 aa, synthetic construct
NRSAGGSSNDNH

SEQ ID NO: 197 - protein, 12 aa, synthetic construct
NDTSGGSSNDNA

SEQ ID NO: 198 - protein, 11 aa, synthetic construct
SKSTGSSNDNA

SEQ ID NO: 199 - protein, 11 aa, synthetic construct
SHTTGATNDNT

SEQ ID NO: 200 - protein, 11 aa, synthetic construct
NNSNGATNDNT

SEQ ID NO: 201 - protein, 12 aa, synthetic construct
NKSAGGATNDNN

SEQ ID NO: 202 - protein, 12 aa, synthetic construct
NGTDGGSTNDNN

SEQ ID NO: 203 - protein, 11 aa, synthetic construct
NKSTGSTNDNH

SEQ ID NO: 204 - protein, 11 aa, synthetic construct
STTTGSTNDNT

SEQ ID NO: 205 - protein, 12 aa, synthetic construct
NRTTGGATNDNA

SEQ ID NO: 206 - protein, 12 aa, synthetic construct
NGTNGGSTNDNH

SEQ ID NO: 207 - protein, 12 aa, synthetic construct
SKTTGGASNDNT

SEQ ID NO: 208 - protein, 12 aa, synthetic construct
NNTAGGSSNDNA

SEQ ID NO: 209 - protein, 12 aa, synthetic construct
SSTNGGSSNDNH

SEQ ID NO: 210 - protein, 12 aa, synthetic construct
SNTSGGATNDNA

SEQ ID NO: 211 - protein, 11 aa, synthetic construct
STTSGATNDNT

SEQ ID NO: 212 - protein, 12 aa, synthetic construct
SHTSGGSSNDNN

SEQ ID NO: 213 - protein, 11 aa, synthetic construct
NETSGSSNDNA

SEQ ID NO: 214 - protein, 12 aa, synthetic construct
STSAGGASNDNH

SEQ ID NO: 215 - protein, 11 aa, synthetic construct
STTTGSSNDNN

SEQ ID NO: 216 - protein, 12 aa, synthetic construct
SKSAGGSSNDNN

SEQ ID NO: 217 - protein, 11 aa, synthetic construct
NHSTGATNDNA

SEQ ID NO: 218 - protein, 12 aa, synthetic construct
NNSTGGATNDNA

SEQ ID NO: 219 - protein, 12 aa, synthetic construct
STSAGGSTNDNP

SEQ ID NO: 220 - protein, 12 aa, synthetic construct
SKSGGGATNDNA

SEQ ID NO: 221 - protein, 11 aa, synthetic construct
SKSDGASNDNA

SEQ ID NO: 222 - protein, 12 aa, synthetic construct
NNTSGGSTNDNN

SEQ ID NO: 223 - protein, 12 aa, synthetic construct
STSNGGSTNDNH

SEQ ID NO: 224 - protein, 12 aa, synthetic construct
SDSSGGSTNDNH

SEQ ID NO: 225 - protein, 12 aa, synthetic construct
NATAGGSSNDNH

SEQ ID NO: 226 - protein, 11 aa, synthetic construct
SNTDGSSNDNT

SEQ ID NO: 227 - protein, 11 aa, synthetic construct
NSTDGATNDNA

SEQ ID NO: 228 - protein, 10 aa, synthetic construct
SESGSTNDNH

SEQ ID NO: 229 - protein, 11 aa, synthetic construct
NQTSGATNDNA

SEQ ID NO: 230 - protein, 11 aa, synthetic construct
NQTDGASNDNP

SEQ ID NO: 231 - protein, 11 aa, synthetic construct
NKTSGASNDNA

SEQ ID NO: 232 - protein, 11 aa, synthetic construct
SKTAGSSNDNA

SEQ ID NO: 233 - protein, 11 aa, synthetic construct
SGTNGSSNDNA

SEQ ID NO: 234 - protein, 12 aa, synthetic construct
NRTTGGATNDNP

SEQ ID NO: 235 - protein, 12 aa, synthetic construct
SSTGGGSSNDNN

SEQ ID NO: 236 - protein, 12 aa, synthetic construct
SSSDGGATNDNT

SEQ ID NO: 237 - protein, 12 aa, synthetic construct
NGSNGGSSNDNN

SEQ ID NO: 238 - protein, 11 aa, synthetic construct
SQSAGSSNDNA

SEQ ID NO: 239 - protein, 12 aa, synthetic construct
NQTAGGSSNDNH

SEQ ID NO: 240 - protein, 12 aa, synthetic construct
SETGGGSSNDNP

SEQ ID NO: 241 - protein, 11 aa, synthetic construct
STTDGSTNDNT

SEQ ID NO: 242 - protein, 12 aa, synthetic construct
NQSSGGSSNDNH

SEQ ID NO: 243 - protein, 12 aa, synthetic construct
SESNGGSTNDNH

SEQ ID NO: 244 - protein, 11 aa, synthetic construct
SATNGSTNDNH

SEQ ID NO: 245 - protein, 11 aa, synthetic construct
SESSGSSNDNA

SEQ ID NO: 246 - protein, 12 aa, synthetic construct
SDTTGGSTNDNN

SEQ ID NO: 247 - protein, 11 aa, synthetic construct
SESSGASNDNA

SEQ ID NO: 248 - protein, 12 aa, synthetic construct
SQTDGGASNDNT

SEQ ID NO: 249 - protein, 11 aa, synthetic construct
NQTDGASNDNA

SEQ ID NO: 250 - protein, 12 aa, synthetic construct
NKSAGGATNDNT

SEQ ID NO: 251 - protein, 11 aa, synthetic construct
SKSSGASNDNN

SEQ ID NO: 252 - protein, 11 aa, synthetic construct
NSSSGSTNDNH

SEQ ID NO: 253 - protein, 11 aa, synthetic construct
SKTTGASNDNA

SEQ ID NO: 254 - protein, 11 aa, synthetic construct
STSTGASNDNT

SEQ ID NO: 255 - protein, 12 aa, synthetic construct
NHSNGGASNDNN

SEQ ID NO: 256 - protein, 12 aa, synthetic construct
SESSGGATNDNA

SEQ ID NO: 257 - protein, 12 aa, synthetic construct
NNTTGGATNDNA

SEQ ID NO: 258 - protein, 12 aa, synthetic construct
SKTTGGASNDNN

SEQ ID NO: 259 - protein, 12 aa, synthetic construct
STTGGGATNDNP

SEQ ID NO: 260 - protein, 10 aa, synthetic construct
STSGATNDNA

SEQ ID NO: 261 - protein, 11 aa, synthetic construct
SKTSGSTNDNN

SEQ ID NO: 262 - protein, 11 aa, synthetic construct
SATTGSTNDNP

SEQ ID NO: 263 - protein, 11 aa, synthetic construct
SKTSGATNDNP

SEQ ID NO: 264 - protein, 12 aa, synthetic construct
SNTTGGASNDNP

SEQ ID NO: 265 - protein, 12 aa, synthetic construct
NKSAGGSTNDNH

SEQ ID NO: 266 - protein, 11 aa, synthetic construct
NHSAGATNDNT

SEQ ID NO: 267 - protein, 11 aa, synthetic construct
NRSDGSTNDNA

SEQ ID NO: 268 - protein, 12 aa, synthetic construct
SDTDGGSSNDNP

SEQ ID NO: 269 - protein, 11 aa, synthetic construct
SQSTGATNDNP

SEQ ID NO: 270 - protein, 12 aa, synthetic construct
NGTAGGSSNDNN

SEQ ID NO: 271 - protein, 12 aa, synthetic construct
SGTAGGSSNDNA

SEQ ID NO: 272 - protein, 12 aa, synthetic construct
SGSNGGSTNDNT

SEQ ID NO: 273 - protein, 12 aa, synthetic construct
NKSSGGATNDNH

SEQ ID NO: 274 - protein, 12 aa, synthetic construct
SRTNGGSSNDNH

SEQ ID NO: 275 - protein, 11 aa, synthetic construct
SSTTGASNDNP

SEQ ID NO: 276 - protein, 12 aa, synthetic construct
SKTAGGASNDNA

SEQ ID NO: 277 - protein, 12 aa, synthetic construct
SDSTGGSSNDND

SEQ ID NO: 278 - protein, 11 aa, synthetic construct
NRSTGASNDNH

SEQ ID NO: 279 - protein, 12 aa, synthetic construct
NASDGGSTNDNT

SEQ ID NO: 280 - protein, 12 aa, synthetic construct
NETAGGSSNDND

SEQ ID NO: 281 - protein, 11 aa, synthetic construct
NESGGATNDNH

SEQ ID NO: 282 - protein, 11 aa, synthetic construct
NNTDGSSNDNN

SEQ ID NO: 283 - protein, 12 aa, synthetic construct
NKSAGGATNDNH

SEQ ID NO: 284 - protein, 12 aa, synthetic construct
NHSTGGSSNDNH

SEQ ID NO: 285 - protein, 12 aa, synthetic construct
SSSTGGATNDNP

SEQ ID NO: 286 - protein, 10 aa, synthetic construct
SQSGATNDNP

SEQ ID NO: 287 - protein, 12 aa, synthetic construct
STTNGGSSNDNA

SEQ ID NO: 288 - protein, 12 aa, synthetic construct
SRTGGGSSNDNA

SEQ ID NO: 289 - protein, 10 aa, synthetic construct
SRSGSTNDNH

SEQ ID NO: 290 - protein, 12 aa, synthetic construct
NQTTGGATNDNP

SEQ ID NO: 291 - protein, 10 aa, synthetic construct
NPSGATNDNN

SEQ ID NO: 292 - protein, 12 aa, synthetic construct
NRTNGGSTNDNP

SEQ ID NO: 293 - protein, 12 aa, synthetic construct
SQSTGGSSNDNP

SEQ ID NO: 294 - protein, 11 aa, synthetic construct
SSTGGSTNDNT

SEQ ID NO: 295 - protein, 12 aa, synthetic construct
NASTGGSTNDNH

SEQ ID NO: 296 - protein, 11 aa, synthetic construct
SSTSGASNDNH

SEQ ID NO: 297 - protein, 11 aa, synthetic construct
SKTNGATNDNT

SEQ ID NO: 298 - protein, 12 aa, synthetic construct
NSSAGGATNDNP

SEQ ID NO: 299 - protein, 12 aa, synthetic construct
NQSTGGATNDNP

SEQ ID NO: 300 - protein, 11 aa, synthetic construct
NNSAGSSNDNP

SEQ ID NO: 301 - protein, 12 aa, synthetic construct
SPTNGGSTNDNP

SEQ ID NO: 302 - protein, 11 aa, synthetic construct
NASSGSTNDNA

SEQ ID NO: 303 - protein, 12 aa, synthetic construct
NQSDGGSTNDNN

SEQ ID NO: 304 - protein, 12 aa, synthetic construct
NQTSGGASNDNP

SEQ ID NO: 305 - protein, 12 aa, synthetic construct
NASDGGSTNDNN

SEQ ID NO: 306 - protein, 11 aa, synthetic construct
SDTAGATNDNP

SEQ ID NO: 307 - protein, 11 aa, synthetic construct
SATTGSTNDNA

SEQ ID NO: 308 - protein, 11 aa, synthetic construct
NNTGGATNDNA

SEQ ID NO: 309 - protein, 11 aa, synthetic construct
NQTDGSTNDND

SEQ ID NO: 310 - protein, 11 aa, synthetic construct
NNTGGATNDNP

SEQ ID NO: 311 - protein, 12 aa, synthetic construct
SETGGGSSNDNA

SEQ ID NO: 312 - protein, 12 aa, synthetic construct
NPTSGGSTNDNN

SEQ ID NO: 313 - protein, 12 aa, synthetic construct
SPSDGGSSNDNT

SEQ ID NO: 314 - protein, 10 aa, synthetic construct
NETGSSNDNA

SEQ ID NO: 315 - protein, 11 aa, synthetic construct
NTTSGSSNDNN

SEQ ID NO: 316 - protein, 11 aa, synthetic construct
STTNGSTNDNA

SEQ ID NO: 317 - protein, 11 aa, synthetic construct
NQSNGSTNDNN

SEQ ID NO: 318 - protein, 11 aa, synthetic construct
NETGGSTNDNN

SEQ ID NO: 319 - protein, 12 aa, synthetic construct
NKSAGGSSNDNH

SEQ ID NO: 320 - protein, 12 aa, synthetic construct
NSSNGGASNDNP

SEQ ID NO: 321 - protein, 12 aa, synthetic construct
NSSDGGATNDNT

SEQ ID NO: 322 - protein, 12 aa, synthetic construct
NSTAGGATNDND

SEQ ID NO: 323 - protein, 11 aa, synthetic construct
NQTAGSTNDNH

SEQ ID NO: 324 - protein, 12 aa, synthetic construct
NQTTGGSTNDND

SEQ ID NO: 325 - protein, 12 aa, synthetic construct
NNTAGGASNDNT

SEQ ID NO: 326 - protein, 10 aa, synthetic construct
SHTGASNDNA

SEQ ID NO: 327 - protein, 12 aa, synthetic construct
NASTGGATNDNT

SEQ ID NO: 328 - protein, 11 aa, synthetic construct
NSSNGSTNDNH

SEQ ID NO: 329 - protein, 12 aa, synthetic construct
NATTGGSTNDNN

SEQ ID NO: 330 - protein, 12 aa, synthetic construct
NKSDGGSTNDNN

SEQ ID NO: 331 - protein, 11 aa, synthetic construct
NDSTGASNDNT

SEQ ID NO: 332 - protein, 12 aa, synthetic construct
NGTSGGSTNDND

SEQ ID NO: 333 - protein, 12 aa, synthetic construct
NDTDGGSSNDNA

SEQ ID NO: 334 - protein, 11 aa, synthetic construct
NQTTGSSNDNA

SEQ ID NO: 335 - protein, 10 aa, synthetic construct
SQTGASNDNA

SEQ ID NO: 336 - protein, 12 aa, synthetic construct
STSDGGSTNDNH

SEQ ID NO: 337 - DNA, 26 bp, synthetic construct
  source 1..26: mol_type = other DNA; note = Forward NGS (liver explant)
actgaatggctgggcgacagagtcat

SEQ ID NO: 338 - DNA, 26 bp, synthetic construct
  source 1..26: mol_type = other DNA; note = 2y library (upstream)
acaaccatctctacaagcaaatctcc

SEQ ID NO: 339 - DNA, 24 bp, synthetic construct
  source 1..24: mol_type = other DNA; note = 2y library (downstream)
tactttggctacagcaccccttgg

SEQ ID NO: 340 - protein, 735 aa, synthetic construct
  source 1..735: note = AAVC11.12
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYNKSVSVDFTVDTNGVYSEPRPIGTRYLTRNL

SEQ ID NO: 341 - DNA, 30 bp, synthetic construct
  source 1..30: mol_type = other DNA; note = Codon-optimised 1
agccagagcggcgccagcaacgacaaccac

SEQ ID NO: 342 - DNA, 30 bp, synthetic construct
  source 1..30: mol_type = other DNA; note = Codon-optimised 2
tcccaatccggagcttccaatgataatcat

(19) United States
(12) Patent Application Publication
(10) Pub. No.: US 2024/0352077 A1
(43) Pub. Date: Oct. 24, 2024

(54) MODIFIED AAV CAPSIDS AND VECTORS

(71) Applicant: Children's Medical Research Institute, Westmead (AU)

(72) Inventors: Marti Cabanes Creus, Westmead (AU); Leszek Lisowski, Westmead (AU)

(21) Appl. No.: 18/686,131

(22) PCT Filed: Aug. 25, 2022
(86) PCT No.: PCT/AU2022/051006

§ 371 Date: Feb. 23, 2024

(30) Foreign Application Priority Data
Aug. 25, 2021 (AU) 2021902737

(51) Int. Cl. C07K 14/005; C12N 15/86

(52) U.S. Cl. CPC C07K 14/005; C12N 15/86; C12N 2750/14122; C12N 2750/14143; C12N 2750/14145

(57) ABSTRACT

The present disclosure relates generally to modified adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### FIELD OF THE DISCLOSURE

The present disclosure relates generally to modified adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three structural capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four known regulatory proteins: Rep78, Rep68, Rep52 and Rep40. These Rep proteins are involved in AAV genome replication, packaging, genomic integration and other processes. More recently, an X gene has been identified in the 3′ end of the AAV2 genome (Cao et al. PLoS One, 2014, 9:e104596). The encoded X protein appears to be involved in the AAV life cycle, including DNA replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contain one or more heterologous sequences flanked by the ITRs, have been successfully used in gene therapy settings. These AAV vectors are widely used to deliver heterologous nucleic acid to cells of a subject for therapeutic purposes, and in many instances, it is the expression of the heterologous nucleic acid that imparts the therapeutic effect. Although several AAV vectors have now been used in the clinic, there are a limited number that exhibit the required in vivo transduction efficiency of primary human cells/tissues to facilitate adequate expression of the heterologous nucleic acid for therapeutic applications. There is therefore a need to develop alternative AAV vectors that contain capsid proteins that facilitate efficient transduction of host cells in vivo.

### SUMMARY OF THE DISCLOSURE

The present disclosure is predicated in part on the generation and identification of AAV capsid variant variable region (VR)-I sequences that are associated with improved transduction efficiency. In particular embodiments, capsid polypeptides of the present disclosure that comprise a variant VR-I sequence described herein facilitate efficient transduction of human cells (such as human hepatocytes) when contained in an AAV vector. Typically, the in vivo transduction of AAV vectors comprising a capsid polypeptide of the present disclosure is improved compared to AAV vectors comprising other AAV capsid polypeptides (e.g. the prototypic AAV2 capsid set forth in SEQ ID NO:5). The capsids polypeptides of the present disclosure are therefore particularly useful in preparing AAV vectors, and in particular, AAV vectors for gene therapy uses. Similarly, AAV vectors comprising a capsid polypeptide of the present disclosure (i.e. having a capsid comprising or consisting of a capsid polypeptide of the present disclosure) are of particular use in gene therapy applications, such as for delivery of heterologous nucleic acids for the treatment of various diseases and conditions.

In one aspect, the disclosure provides a capsid polypeptide comprising a variant VR-I, wherein the variant VR-I comprises a sequence set forth in any one of SEQ ID NOs: 21-336, and wherein the capsid polypeptide comprises a sequence having at least or about 85%, 86%, 87%, 88%, 89%, 90%, 91%, 92%, 93%, 94%, 95%, 96%, 97%, 98% or 99% sequence identity to: (i) the sequence set forth in SEQ ID NO:2; (ii) the sequence of amino acids at positions 138-735 of SEQ ID NO:2; or (iii) the sequence of amino acids at positions 203-736 of SEQ ID NO:2.

In a particular embodiment, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Another aspect of the disclosure provides a capsid polypeptide comprising a variant VR-I, wherein the variant VR-I comprises a sequence set forth in any one of SEQ ID NOs: 21-336, and wherein the capsid polypeptide comprises a sequence having at least or about 85%, 86%, 87%, 88%, 89%, 90%, 91%, 92%, 93%, 94%, 95%, 96%, 97%, 98% or 99% sequence identity to: (i) the sequence set forth in SEQ ID NO: 4; (ii) the sequence of amino acids at positions 138-735 of SEQ ID NO:4; or (iii) the sequence of amino acids at positions 204-735 of SEQ ID NO:4.

In a particular embodiment, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Another aspect of the disclosure provides a capsid polypeptide comprising a variant VR-I, wherein the variant VR-I comprises a sequence set forth in any one of SEQ ID NOs: 21-336, and wherein the capsid polypeptide comprises a sequence having at least or about 85%, 86%, 87%, 88%, 89%, 90%, 91%, 92%, 93%, 94%, 95%, 96%, 97%, 98% or 99% sequence identity to: (i) the sequence set forth in SEQ ID NO: 340; (ii) the sequence of amino acids at positions 138-735 of SEQ ID NO: 340; or (iii) the sequence of amino acids at positions 204-735 of SEQ ID NO:340.

In a particular embodiment, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Also provided is an AAV vector, comprising a capsid polypeptide of the present disclosure. In some examples, the AAV vector comprises a heterologous coding sequence, such as a heterologous coding sequence that encodes a peptide, polypeptide or polynucleotide (e.g. a therapeutic peptide, polypeptide or polynucleotide).

Also provided is a nucleic acid molecule encoding a capsid polypeptide of the present disclosure. In another aspect, provided is a vector comprising the aforementioned nucleic acid molecule. In some examples, the vector is selected from among a plasmid, cosmid, phage and transposon.

In another aspect, provided is a host cell, comprising an AAV vector, nucleic acid molecule or vector of the present disclosure.

Also provided is a method for introducing a heterologous coding sequence into a host cell, comprising contacting a host cell with an AAV vector of the present disclosure. In some examples, the host cell is a hepatocyte. In particular embodiments, contacting a host cell with the AAV vector comprises administering the AAV vector to a subject. In other embodiments, the method is in vitro or ex vivo.

In another aspect, provided is a method for producing an AAV vector, comprising culturing a host cell comprising a nucleic acid molecule encoding the capsid polypeptide of the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide of the present disclosure, wherein the capsid encapsidates the heterologous coding sequence. In some examples, the host cell is a hepatocyte.

Also provided is a method for producing a modified AAV vector that exhibits enhanced transduction efficiency in a human hepatocyte, comprising: a) identifying a reference capsid polypeptide for transducing human hepatocytes in vivo; b) modifying the sequence of the reference capsid polypeptide at one or more of positions 262, 263, 264, 265, 266, 267, 268, 269, 270 and 271, with numbering relative to SEQ ID NO:5, that comprises a sequence set forth in any one of SEQ ID NOs: 21-336 at positions 262-271, with numbering relative to SEQ ID NO:5; and c) vectorising the modified capsid polypeptide to thereby produce a modified AAV vector. In some examples, wherein the reference capsid polypeptide comprises at least or about 85%, 86%, 87%, 88%, 89%, 90%, 91%, 92%, 93%, 94%, 95%, 96%, 97%, 98%, or 99% sequence identity to the sequence set forth in SEQ ID NO:2, 4 or 340.

In some embodiments, the method further comprises assessing the transduction efficiency of the modified AAV vector in an in vivo system that utilises human hepatocytes. In one example, the in vivo system comprises a small animal (e.g. a mouse) with a chimeric liver comprising human hepatocytes, such as a hFRG mouse.

### BRIEF DESCRIPTION OF THE DRAWINGS

Embodiments of the disclosure are described herein, by way of non-limiting example only, with reference to the following drawings.

FIG. 1 is an alignment of the capsid VR-I of various AAV serotypes. The first alanine in the alignment corresponds to A248 in AAV2. The VR-I spans positions 262-271 of the AAV2 capsid. S262 of AAV2 corresponds to S262 in AAV1 and AAV3b, N262 in AAV9, S263 in AAV7, and N263 in AAV8 and AAV10. Thus, VR-I spans positions 262-271 of the AAV3b capsid, positions 262-272 of the AAV1 capsid, positions 262-273 of the AAV9 capsid, positions 263-273 of the AAV7 capsid, and positions 263-274 of the AAV8 and AAV10 capsids.

FIG. 2 is an alignment of the capsid sequences from AAV2, AAVC11.11 and AAV-LK03-REDH. The shaded, bolded text represents the VR-I, as defined for the purposes of the present disclosure.

FIG. 3 shows in vivo performance of variants from secondary libraries as described in Example 4 (no IVIg pre-immunization). (A) DNA uptake in human hepatocytes, and (B) cDNA expression (functional transduction) in human hepatocytes). n=3 FRG mice with 2 BC per capsid. Variants are as shown in Tables 14, 16 and 19.

FIG. 4 shows in vivo performance of variants from secondary libraries as described in Example 4 (IVIg pre-immunization). (A) DNA uptake in human hepatocytes, and (B) cDNA expression (functional transduction) in human hepatocytes). n=3 FRG mice with 2 BC per capsid. Variants are as shown in Tables 14, 16 and 19.

FIG. 5 shows in vivo performance of variants from secondary libraries as described in Example 4 (no IVIg pre-immunization). (A) DNA uptake in human hepatocytes, and (B) cDNA expression (functional transduction) in human hepatocytes). n=3 FRG mice with 2 BC per capsid. Variants are as shown in Tables 15, 17, 18 and 20.

FIG. 6 shows in vivo performance of variants from secondary libraries as described in Example 4 (IVIg pre-immunization). (A) DNA uptake in human hepatocytes, and (B) cDNA expression (functional transduction) in human hepatocytes). n=3 FRG mice with 2 BC per capsid. Variants are as shown in Tables 15, 17, 18 and 20.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or “AAV vector” refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic, bioengineered or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the bioengineered DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, the term “transduction” refers to entry of AAV vector into one or more particular cell types and transferal of the DNA contained within the AAV vector into the cell. Transduction can be assessed by measuring the amount of AAV DNA or RNA expressed from the AAV DNA in a cell or population of cells, and/or by assessing the number of cells in a population that contain AAV DNA or RNA expressed from the DNA. Where the presence or amount of RNA is assessed, the type of transduction assessed is referred to herein as “functional transduction”, i.e. the ability of the AAV to transfer DNA to the cell and have that DNA expressed. The term “transduction efficiency” and grammatical variations thereof refers to the ability of an AAV vector to transduce host cells, and more particularly the efficiency with which an AAV vector transduces host cells. In particular embodiment, the transduction efficiency is in vivo transduction efficiency, and refers to the ability of an AAV vector to transduce host cells in vivo following administration of the vector to the subject. Transduction efficiency can be assessed in a number of ways known to those in the art, including assessing the number of host cells transduced following exposure to, or administration of, a given number of vector particles (e.g. as assessed by expression of a reporter gene from the vector genome, such as GFP or eGFP, using microscopy or flow cytometry techniques); the amount of vector DNA (e.g. number of vector genomes) in a population of host cells following exposure to a given number of vector particles; the amount of vector RNA in population of host cells following exposure to a given number of vector particles; and the level of protein expression from a reporter gene (e.g. GFP or eGFP) in the vector genome in a population of host cells following exposure to, or administration of, a given number of vector particles. The population of host cells can represent a particular number of host cells, a volume or weight of tissue, or an entire organ (e.g. liver). In vivo transduction efficiency can reflect the ability of an AAV vector to access host cells, such as hepatocytes in the liver; the ability of an AAV vector to enter host cells; and/or expression of a heterologous coding sequence contained in the vector genome upon host cell entry.

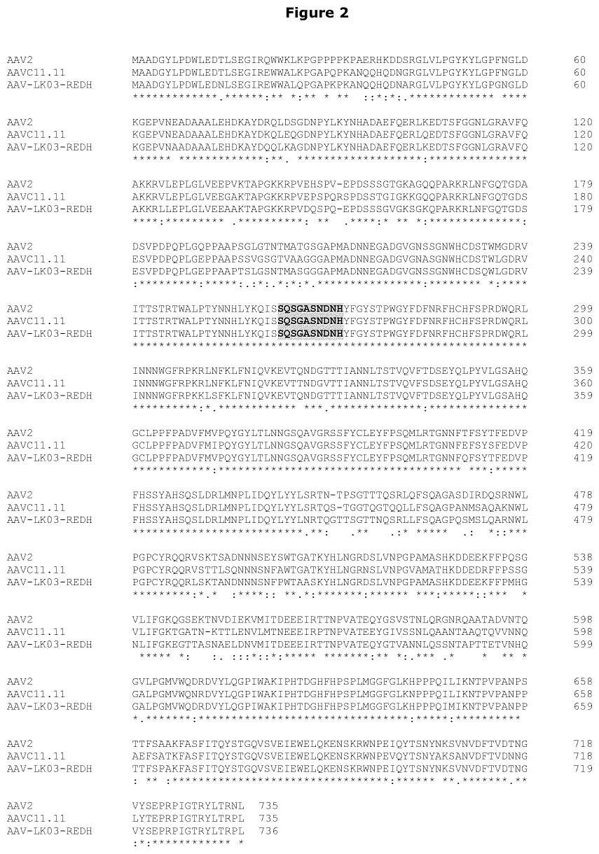

As used herein, “corresponding nucleotides”, “corresponding amino acid residues” or “corresponding positions” refer to nucleotides, amino acids or positions that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc) and others known to those of skill in the art. By aligning the sequences of polynucleotides, one skilled in the art can identify corresponding nucleotides. By aligning two AAV capsid polypeptides (e.g. as shown in FIG. 2), one of skill in the art can identify regions or amino acids residues within one AAV polypeptide that correspond to various regions or residues in the other AAV polypeptide. For example, the serine at position 262 of the AAV2 capsid polypeptide set forth in SEQ ID NO:5 is the corresponding amino acid of, or corresponds to, the serine at position 263 of the AAVC11.11 capsid polypeptide set forth in SEQ ID NO:4 (as shown in the alignment in FIG. 2). In another example, and also with reference to FIG. 2, position 734 of the AAVC11.11 capsid polypeptide aligns with, or corresponds to, position 735 of the AAV-LK03-REDH capsid polypeptide, and the asparagine at position 734 of the AAV2 capsid polypeptide corresponds to, or is the corresponding amino acid of, the proline at position 735 of the AAV-LK03-REDH capsid polypeptide. In a further example, residues at positions 262-271 of the AAV2 capsid polypeptide, which make up the VR-I of the AAV2 capsid, correspond to residues 263-273 of the AAV7 capsid polypeptide (i.e. the VR-I of the AAV7 capsid) and residues 263-272 of the AAVC11.11 capsid polypeptide (i.e. the VR-I of the AAVC11.11 capsid polypeptide). Thus, when amino acid residues or positions are referred to herein with respect to a particular capsid polypeptide, it is understood that, where appropriate, the reference is also to the corresponding amino acid residue or position in another capsid polypeptide. For example, reference to a capsid polypeptide comprising “a VR-I comprising the sequence SQSGASNDNH (SEQ ID NO: 12)” encompasses not only the AAV2 and AAVC11.11 capsid polypeptides set forth in SEQ ID NO:5 and 4, respectively, which have a VR-I having the sequence SQSGASNDNH (SEQ ID NO: 12) at positions 262-271, but also other capsid polypeptides having a VR-I comprising the sequence SQSGASNDNH at positions that correspond to positions 262-271 of SEQ ID NO:5, such as the AAV-LK03-REDH capsid polypeptide set forth in SEQ ID NO:2 (where the VR-I comprising the sequence SQSGASNDNH is at positions 263-272), on the understanding that as defined herein, the VR-I of any AAV capsid polypeptide includes residues corresponding to those at positions 262-271 of the AAV2 capsid set forth in SEQ ID NO:5. In another example, reference to a capsid polypeptide comprising “a VR-I comprising the sequence NRTGGGATNDNA (SEQ ID NO:23)” encompasses capsid polypeptides having the sequence NRTGGGATNDNA (SEQ ID NO:23) at positions that correspond to positions 262-271 of SEQ ID NO:5. As would be appreciated, because the sequence NRTGGGATNDNA (SEQ ID NO: 23) comprises 12 amino acids, the actual position of the residues will be different. For example, if the capsid backbone is the AAV2 capsid set forth in SEQ ID NO:5, then the capsid polypeptide will comprise the sequence NRTGGGATNDNA (SEQ ID NO:23) at positions 262-273 (which correspond to positions 262-271 of the AAV2 capsid set forth in SEQ ID NO:5).

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “host cell” refers to a cell, such as a mammalian cell, that has introduced into it the exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

As used herein, the term “conservative sequence modifications” or “conservative substitution” refers to amino acid modifications that do not significantly affect or alter the characteristics of a vector containing the amino acid sequence. Such conservative modifications include amino acid substitutions, additions and deletions. Modifications can be introduced into a vector that are compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a capsid can be replaced with other amino acid residues from the same side chain family and the altered capsid can be tested for tropism and/or the ability to deliver a payload using the functional assays described herein.

TABLE 1
IUPAC nucleotide codes

| IUPAC nucleotide code | Base |
|---|---|
| A | Adenine |
| C | Cytosine |
| G | Guanine |
| T | Thymine |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

It will be appreciated that the above described terms and associated definitions are used for the purpose of explanation only and are not intended to be limiting.

### Capsid Polypeptides

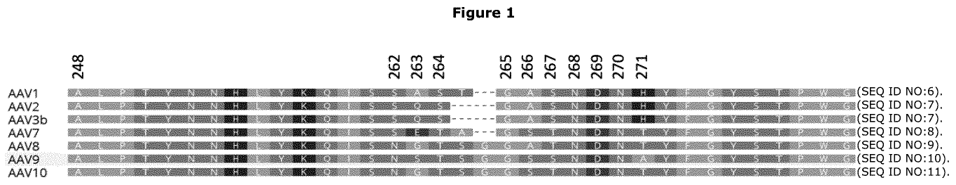

The present disclosure is predicated in part on the identification of variant capsid VR-I sequences that impart improved transduction properties on an AAV vector. Thus, provided herein are capsid polypeptides, including isolated capsid polypeptides, comprising the variant VR-I sequences. For the purposes of the present disclosure, the VR-I is defined as including residues at positions that correspond to positions 262-271 of the prototypic AAV2 capsid polypeptide set forth in SEQ ID NO:5. As shown in FIG. 1, this region includes residues at positions 262-271 of the AAV3b capsid, positions 262-272 of the AAV1 capsid, positions 262-273 of the AAV9 capsid, positions 263-273 of the AAV7 capsid, and positions 263-274 of the AAV8 and AAV10 capsids. As shown in FIG. 2, the VR-I includes residues at positions 263-272 of the AAVC11.11 capsid set forth in SEQ ID NO:4, positions 262-271 of the AAV-LK03-REDH capsid set forth in SEQ ID NO:2, and positions 263-272 of the AAVC11.12 capsid set forth in SEQ ID NO:340.

Typically, the capsid polypeptides, when present in the capsid of an AAV vector, facilitate efficient transduction of human cells (such as human hepatocytes). The in vivo transduction of cells by AAV vectors having a capsid comprising a variant VR-I is generally increased or enhanced compared to AAV vectors comprising a reference AAV capsid polypeptide (e.g. the prototypic AAV2 capsids set forth in SEQ ID NO:5, or the LK03-REDH capsid or AAVC11.11 capsid set forth in SEQ ID NO: 2 or 4, respectively). Transduction or transduction efficiency of AAV vectors can be increased by at least or about 10%, 20%, 30%, 40%, 50%, 60%, 70%, 80%, 90%, 100%, 200%, 300%, 400%, 500%, 600%, 700%, 800%, 900%, 1000% or more, e.g. an AAV vector comprising a capsid polypeptide of the present disclosure can be at least or about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to a reference AAV capsid polypeptide. In particular examples, the increased transduction or transduction efficiency is observed in human liver tissue or human hepatocytes.

The capsid polypeptides of the present disclosure are therefore particularly useful in preparing AAV vectors, and in particular AAV vectors for gene therapy uses. In exemplary embodiments, the capsid polypeptides of the present disclosure are particularly useful in preparing AAV vectors that transduce hepatocytes, and in particular, human hepatocytes, and are thus useful for gene therapy applications targeting the liver.

The capsid polypeptides of the present disclosure comprise a variant VR-I comprising a sequence represented by X₁X₂X₃X₄X₅X₆X₇X₈X₉X₁₀X₁₁X₁₂, where X₁ is S or N; X₂ is Q, S, A, G, E, D, H, K, N, P, T or R; X₃ is S or T; X₄ is A, S or T or no amino acid; X₅ is G or no amino acid; X₆ is G; X₇ is A or S; X₈ is S or T; X₉ is N; X₁₀ is D; X₁₁ is N; and X₁₂ is T, A or H, or a sequence having 1, 2 or 3 conservative amino acid substitutions of the sequence represented by X₁X₂X₃X₄X₅X₆X₇X₈X₉X₁₀X₁₁X₁₂. In particular examples, the variant VR-I comprises a sequence set forth in any one of SEQ ID NOs: 21-336 (see Table 2) or a sequence having 1, 2 or 3 conservative amino acid substitutions thereof. In particular examples, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32, or a sequence having 1, 2 or 3 conservative amino acid substitutions thereof. As would be appreciated, the variant VR-I is present in the capsid polypeptide at positions corresponding to positions 262-271 of the prototypic AAV2 capsid polypeptide set forth in SEQ ID NO:5 (i.e. at positions 262-271 of the capsid polypeptide, with numbering relative to the prototypic AAV2 capsid polypeptide set forth in SEQ ID NO:5).

TABLE 2
Variant VR-I sequences

| SEQ | |
|---|---|
| ID NO | Sequence |
| 21 | SRSAGGASNDNA |
| 22 | SRSSGGSTNDNA |
| 23 | NRTGGGATNDNA |
| 24 | NTTAGSSNDNP |
| 25 | NRTAGGSTNDNH |
| 26 | SRSSGGSSNDNT |
| 27 | SKSTGASNDNN |
| 28 | SRSSGGASNDNH |
| 29 | NKTGGGASNDNT |
| 30 | NKTNGGATNDNH |
| 31 | NRTTGGATNDNH |
| 32 | NKTGGASNDNT |
| 33 | SKSAGGSSNDNA |
| 34 | NRTSGGASNDNH |
| 35 | SRSTGGASNDNA |
| 36 | SKSTGASNDNA |
| 37 | NKTAGGATNDNT |
| 38 | NRSSGGATNDNA |
| 39 | SRSSGGASNDNA |
| 40 | SKTTGGSSNDNA |
| 41 | SKSAGGASNDNH |
| 42 | SRSTGGASNDNN |
| 43 | SRSTGGSSNDNA |
| 44 | NKTAGGSTNDNH |
| 45 | NRTNGGATNDNT |
| 46 | NRTTGGSTNDNN |
| 47 | NKTTGGATNDNH |
| 48 | NNSGATNDNH |
| 49 | NHSNGATNDNN |
| 50 | SKTSGSSNDNH |
| 51 | SRSAGGSSNDNT |
| 52 | NGTAGSSNDNN |
| 53 | NRTSGGASNDNA |
| 54 | SSSSGGASNDNN |
| 55 | SRSSGGSSNDNA |
| 56 | NKTNGGASNDNN |
| 57 | NKTGGGASNDNH |
| 58 | SRTTGGSSNDNH |
| 59 | SRTAGGATNDNN |
| 60 | SRSGGGSSNDNT |
| 61 | NHTSGSSNDNH |
| 62 | SKSTGSTNDNH |
| 63 | NSTGGSSNDNA |
| 64 | NESGGGASNDNN |
| 65 | NNSSGATNDNN |
| 66 | NRTNGGATNDNH |
| 67 | NRTGGGATNDNH |
| 68 | NASTGGATNDNN |
| 69 | SDTNGATNDNN |
| 70 | SASGSSNDNH |
| 71 | NDSTGGATNDNN |
| 72 | NATTGGASNDNA |
| 73 | SKTNGSSNDNH |
| 74 | STTSGASNDNA |
| 75 | SHTAGGSSNDNH |
| 76 | STSAGASNDNH |
| 77 | NRSGGATNDNH |
| 78 | NTSGGSSNDNN |
| 79 | NHSAGGASNDNA |
| 80 | SETSGGSSNDNH |
| 81 | SASNGGSSNDNA |
| 82 | SDTGGGASNDNT |
| 83 | SSSGGGATNDNA |
| 84 | SNSAGGSSNDNA |
| 85 | SSTNGGSTNDNH |
| 86 | SGTGGGASNDNA |
| 87 | SGTTGGATNDNN |
| 88 | SGTSGGASNDNA |
| 89 | NDSSGSSNDNT |
| 90 | NKSTGASNDNN |
| 91 | NQTGGSSNDNA |
| 92 | SNSGATNDNH |
| 93 | SHSSGGSSNDNH |
| 94 | SQTGGGASNDNH |
| 95 | SKTSGSSNDNN |
| 96 | SRSSGGASNDNT |
| 97 | NRSTGASNDNN |
| 98 | NGSNGGASNDNN |
| 99 | SRTNGGSTNDNA |
| 100 | SSSGGSTNDNA |
| 101 | SGTNGGSTNDNT |
| 102 | SRSDGGSTNDNN |
| 103 | SGSNGGASNDNN |
| 104 | NRSNGGSSNDNH |
| 105 | SKTNGGASNDNH |
| 106 | SRSSGGATNDNA |
| 107 | SSSTGGATNDNA |
| 108 | NDTGGGATNDNP |
| 109 | SGSGGGSTNDNA |
| 110 | SSSGGGSTNDNA |
| 111 | SDTSGATNDNA |
| 112 | NASTGGATNDNP |
| 113 | NKTSGGSSNDNA |
| 114 | NATGGGATNDNP |
| 115 | SKTNGGSTNDNA |
| 116 | SGTNGGSSNDNT |
| 117 | NTTAGGSTNDNN |
| 118 | SQTGGGASNDNT |
| 119 | NTTTGATNDNT |
| 120 | SHTAGSSNDNA |
| 121 | SKSSGSSNDNA |
| 122 | SSSDGGSTNDNA |
| 123 | SNTTGGSTNDNH |
| 124 | NQTGGGATNDNT |
| 125 | NRSDGGSTNDNN |
| 126 | SGTGGGASNDNH |
| 127 | SNSSGGSTNDNA |
| 128 | NSTNGGATNDNA |
| 129 | NSSSGGATNDNT |
| 130 | SQSSGGATNDNH |
| 131 | SKSDGGSTNDNH |
| 132 | SNTAGGSSNDNT |
| 133 | NESAGGSSNDNN |
| 134 | NHSGGGATNDNP |
| 135 | SSSSGGATNDNA |
| 136 | SHTDGSTNDNH |
| 137 | SATDGGSTNDNA |
| 138 | SKSSGATNDNT |
| 139 | NRSTGGSTNDNT |
| 140 | SPTDGGSTNDNN |
| 141 | SESAGGSTNDNN |
| 142 | NSSGGGSTNDNT |
| 143 | NDTSGGATNDNT |
| 144 | NNTGSSNDNH |
| 145 | NKSSGGSTNDNN |
| 146 | STTSGGSTNDND |
| 147 | SHSAGGSTNDNA |
| 148 | SDTAGGSTNDNN |
| 149 | SRTDGSTNDNP |
| 150 | NHSGATNDNP |
| 151 | NSSGGATNDNA |
| 152 | NNSNGSSNDNA |
| 153 | NPSNGGSSNDND |
| 154 | SQTNGGSTNDND |
| 155 | SDSGSSNDNT |
| 156 | NESAGATNDNN |
| 157 | NPSAGGSSNDNN |
| 158 | SSTGATNDNT |
| 159 | SESNGGSSNDNT |
| 160 | SDTNGSTNDNA |
| 161 | NPSGASNDNT |
| 162 | STSGGGSTNDNP |
| 163 | NHSTGGATNDND |
| 164 | NDSGGATNDNT |
| 165 | SHTDGGSSNDNA |
| 166 | SKSSGATNDNA |
| 167 | SKTGSTNDND |
| 168 | SQTGGGSSNDNP |
| 169 | SHTSGSSNDNP |
| 170 | NASSGSSNDND |
| 171 | SSTAGGSTNDNH |
| 172 | NGSDGGSTNDND |
| 173 | STSSGGSSNDNP |
| 174 | SNTGGASNDNA |
| 175 | NDTDGASNDNA |
| 176 | SDTTGATNDNN |
| 177 | SPSGSTNDNN |
| 178 | NRSAGATNDNP |
| 179 | NTSGATNDNT |
| 180 | NRTGGSTNDNA |
| 181 | SETGGATNDNT |
| 182 | SATNGGSSNDNA |
| 183 | SKTDGSSNDNT |
| 184 | NGTSGSTNDNN |
| 185 | NNSSGGSSNDNA |
| 186 | NQSTGGSSNDNT |
| 187 | NQSNGSTNDNA |
| 188 | SSTTGSTNDNA |
| 189 | NDSGGSTNDNT |
| 190 | NETSGGSSNDNA |
| 191 | NSTDGATNDNP |
| 192 | NRSSGGSTNDNH |
| 193 | SKTGGATNDNA |
| 194 | NKSTGGATNDNT |
| 195 | NSSTGSSNDNA |
| 196 | NRSAGGSSNDNH |
| 197 | NDTSGGSSNDNA |
| 198 | SKSTGSSNDNA |
| 199 | SHTTGATNDNT |
| 200 | NNSNGATNDNT |
| 201 | NKSAGGATNDNN |
| 202 | NGTDGGSTNDNN |
| 203 | NKSTGSTNDNH |
| 204 | STTTGSTNDNT |
| 205 | NRTTGGATNDNA |
| 206 | NGTNGGSTNDNH |
| 207 | SKTTGGASNDNT |
| 208 | NNTAGGSSNDNA |
| 209 | SSTNGGSSNDNH |
| 210 | SNTSGGATNDNA |
| 211 | STTSGATNDNT |
| 212 | SHTSGGSSNDNN |
| 213 | NETSGSSNDNA |
| 214 | STSAGGASNDNH |
| 215 | STTTGSSNDNN |
| 216 | SKSAGGSSNDNN |
| 217 | NHSTGATNDNA |
| 218 | NNSTGGATNDNA |
| 219 | STSAGGSTNDNP |
| 220 | SKSGGGATNDNA |
| 221 | SKSDGASNDNA |
| 222 | NNTSGGSTNDNN |
| 223 | STSNGGSTNDNH |
| 224 | SDSSGGSTNDNH |
| 225 | NATAGGSSNDNH |
| 226 | SNTDGSSNDNT |
| 227 | NSTDGATNDNA |
| 228 | SESGSTNDNH |
| 229 | NQTSGATNDNA |
| 230 | NQTDGASNDNP |
| 231 | NKTSGASNDNA |
| 232 | SKTAGSSNDNA |
| 233 | SGTNGSSNDNA |
| 234 | NRTTGGATNDNP |
| 235 | SSTGGGSSNDNN |
| 236 | SSSDGGATNDNT |
| 237 | NGSNGGSSNDNN |
| 238 | SQSAGSSNDNA |
| 239 | NOTAGGSSNDNH |
| 240 | SETGGGSSNDNP |
| 241 | STTDGSTNDNT |
| 242 | NQSSGGSSNDNH |
| 243 | SESNGGSTNDNH |
| 244 | SATNGSTNDNH |
| 245 | SESSGSSNDNA |
| 246 | SDTTGGSTNDNN |
| 247 | SESSGASNDNA |
| 248 | SQTDGGASNDNT |
| 249 | NQTDGASNDNA |
| 250 | NKSAGGATNDNT |
| 251 | SKSSGASNDNN |
| 252 | NSSSGSTNDNH |
| 253 | SKTTGASNDNA |
| 254 | STSTGASNDNT |
| 255 | NHSNGGASNDNN |
| 256 | SESSGGATNDNA |
| 257 | NNTTGGATNDNA |
| 258 | SKTTGGASNDNN |
| 259 | STTGGGATNDNP |
| 260 | STSGATNDNA |
| 261 | SKTSGSTNDNN |
| 262 | SATTGSTNDNP |
| 263 | SKTSGATNDNP |
| 264 | SNTTGGASNDNP |
| 265 | NKSAGGSTNDNH |
| 266 | NHSAGATNDNT |
| 267 | NRSDGSTNDNA |
| 268 | SDTDGGSSNDNP |
| 269 | SQSTGATNDNP |
| 270 | NGTAGGSSNDNN |
| 271 | SGTAGGSSNDNA |
| 272 | SGSNGGSTNDNT |
| 273 | NKSSGGATNDNH |
| 274 | SRTNGGSSNDNH |
| 275 | SSTTGASNDNP |
| 276 | SKTAGGASNDNA |
| 277 | SDSTGGSSNDND |
| 278 | NRSTGASNDNH |
| 279 | NASDGGSTNDNT |
| 280 | NETAGGSSNDND |
| 281 | NESGGATNDNH |
| 282 | NNTDGSSNDNN |
| 283 | NKSAGGATNDNH |
| 284 | NHSTGGSSNDNH |
| 285 | SSSTGGATNDNP |
| 286 | SQSGATNDNP |
| 287 | STTNGGSSNDNA |
| 288 | SRTGGGSSNDNA |
| 289 | SRSGSTNDNH |
| 290 | NQTTGGATNDNP |
| 291 | NPSGATNDNN |
| 292 | NRTNGGSTNDNP |
| 293 | SQSTGGSSNDNP |
| 294 | SSTGGSTNDNT |
| 295 | NASTGGSTNDNH |
| 296 | SSTSGASNDNH |
| 297 | SKTNGATNDNT |
| 298 | NSSAGGATNDNP |
| 299 | NQSTGGATNDNP |
| 300 | NNSAGSSNDNP |
| 301 | SPTNGGSTNDNP |
| 302 | NASSGSTNDNA |
| 303 | NQSDGGSTNDNN |
| 304 | NQTSGGASNDNP |
| 305 | NASDGGSTNDNN |
| 306 | SDTAGATNDNP |
| 307 | SATTGSTNDNA |
| 308 | NNTGGATNDNA |
| 309 | NQTDGSTNDND |
| 310 | NNTGGATNDNP |
| 311 | SETGGGSSNDNA |
| 312 | NPTSGGSTNDNN |
| 313 | SPSDGGSSNDNT |
| 314 | NETGSSNDNA |
| 315 | NTTSGSSNDNN |
| 316 | STTNGSTNDNA |
| 317 | NQSNGSTNDNN |
| 318 | NETGGSTNDNN |
| 319 | NKSAGGSSNDNH |
| 320 | NSSNGGASNDNP |
| 321 | NSSDGGATNDNT |
| 322 | NSTAGGATNDND |
| 323 | NQTAGSTNDNH |
| 324 | NQTTGGSTNDND |
| 325 | NNTAGGASNDNT |
| 326 | SHTGASNDNA |
| 327 | NASTGGATNDNT |
| 328 | NSSNGSTNDNH |
| 329 | NATTGGSTNDNN |
| 330 | NKSDGGSTNDNN |
| 331 | NDSTGASNDNT |
| 332 | NGTSGGSTNDND |
| 333 | NDTDGGSSNDNA |
| 334 | NQTTGSSNDNA |
| 335 | SQTGASNDNA |
| 336 | STSDGGSTNDNH |

The backbone of the capsid polypeptide (i.e. the residues other than the VR-I region) can be from any AAV capsid polypeptide, including any modified AAV capsid polypeptide, such as NP40, NP59 or LK03 (see e.g. Paulk et al. 2018, Mol Ther. 26(1):289-303; and Lisowski et al., 2014, Nature 506:382-386), or any modified capsid polypeptide described in International Patent Application No. PCT/AU2021/050158, such as AAVC11.12. The capsid polypeptide may comprise the full length VP1 (i.e. corresponding to positions 1-735 of the AAV2 capsid set forth in SEQ ID NO:5), or a fragment thereof, such as the VP2 (i.e. corresponding to positions 138-735 of the AAV2 capsid set forth in SEQ ID NO:5) or the VP3 (i.e. corresponding to positions 203-735 of the AAV2 capsid set forth in SEQ ID NO:5).

In one example, the capsid polypeptides of the present disclosure comprise a variant VR-I comprising a sequence set forth in any one of SEQ ID NOs: 21-336 in a C11.11 backbone (i.e. the capsid polypeptide comprises the variant VR-I flanked by residues 1-262 and 273-735 of the C11.11 polypeptide set forth in SEQ ID NO:4, or the VP2 or VP3 fragments thereof) or have a backbone that has at least or about 85%, 86%, 87%, 88%, 90%, 91%, 92%, 93%, 94%, 95%, 96%, 97%, 98%, or 99% sequence identity to the C11.11 backbone represented by residues 1-262 and 273-735 of the C11.11 polypeptide set forth in SEQ ID NO:4, or the VP2 or VP3 fragments thereof. Thus, in some examples, the capsid polypeptide comprises a sequence that has at least or about 85%, 86%, 87%, 88%, 90%, 91%, 92%, 93%, 94%, 95%, 96%, 97%, 98%, or 99% sequence identity to the VP1 protein set forth in SEQ ID NO:4, the VP2 protein set forth at positions 138-735 of SEQ ID NO:4, or the VP3 protein set forth at positions 204-735 of SEQ ID NO:4, wherein the capsid polypeptide comprises a variant VR-I comprising a sequence set forth in any one of SEQ ID NOs: 21-336 (i.e. comprises the sequence set forth in any one of SEQ ID NOs: 21-336 at positions 262-272, with numbering relative to the C11.11 polypeptide set forth in SEQ ID NO:4).

Optionally, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

In another example, the capsid polypeptides of the present disclosure comprise a variant VR-I comprising a sequence set forth in any one of SEQ ID NOs: 21-336 in a LK03-REDH backbone (i.e. the capsid polypeptide comprises the variant VR-I flanked by residues 1-261 and 272-736 of the LK03-REDH polypeptide set forth in SEQ ID NO:2, or the VP2 or VP3 fragments thereof) or in a backbone that has at least or about 85%, 86%, 87%, 88%, 90%, 91%, 92%, 93%, 94%, 95%, 96%, 97%, 98%, or 99% sequence identity to the LK03-REDH backbone represented by residues 1-261 and 272-736 of the LK03-REDH polypeptide set forth in SEQ ID NO:2, or the VP2 or VP3 fragments thereof. Thus, in some examples, the capsid polypeptides comprise a sequence that has at least or about 85%, 86%, 87%, 88%, 90%, 91%, 92%, 93%, 94%, 95%, 96%, 97%, 98%, or 99% sequence identity to the VP1 protein set forth in SEQ ID NO:4, the VP2 protein set forth at positions 138-736 of SEQ ID NO:2, or the VP3 protein set forth at positions 203-736 of SEQ ID NO:2, wherein the capsid polypeptide comprises a variant VR-I comprising a sequence set forth in any one of SEQ ID NOs: 21-336 (i.e. comprises the sequence set forth in any one of SEQ ID NOs: 21-336 at positions 262-271, with numbering relative to the LK03-REDH polypeptide set forth in SEQ ID NO:2).

Optionally, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

In another example, the capsid polypeptides of the present disclosure comprise a variant VR-I comprising a sequence set forth in any one of SEQ ID NOs: 21-336 in a C11.12 backbone (i.e. the capsid polypeptide comprises the variant VR-I flanked by residues 1-262 and 273-735 of the C11.12 polypeptide set forth in SEQ ID NO:340, or the VP2 or VP3 fragments thereof) or have a backbone that has at least or about 85%, 86%, 87%, 88%, 90%, 91%, 92%, 93%, 94%, 95%, 96%, 97%, 98%, or 99% sequence identity to the C11.12 backbone represented by residues 1-262 and 273-735 of the C11.12 polypeptide set forth in SEQ ID NO:340, or the VP2 or VP3 fragments thereof. Thus, in some examples, the capsid polypeptide comprises a sequence that has at least or about 85%, 86%, 87%, 88%, 90%, 91%, 92%, 93%, 94%, 95%, 96%, 97%, 98%, or 99% sequence identity to the VP1 protein set forth in SEQ ID NO:340, the VP2 protein set forth at positions 138-735 of SEQ ID NO:340, or the VP3 protein set forth at positions 204-735 of SEQ ID NO:340, wherein the capsid polypeptide comprises a variant VR-I comprising a sequence set forth in any one of SEQ ID NOs: 21-336 (i.e. comprises the sequence set forth in any one of SEQ ID NOs: 21-336 at positions 262-272, with numbering relative to the C11.12 polypeptide set forth in SEQ ID NO:340).

Optionally, the variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Also provided are nucleic acid molecules, including isolated nucleic acid molecules, encoding a capsid polypeptide described herein. Thus, for example, amongst the nucleic acid molecules provided herein are those encoding the VP1, VP2 and/or VP3 of any one of the capsid polypeptides described herein that comprise a variant VR-I comprising a sequence set forth in any one of SEQ ID NOs: 21-336.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein, such as a polypeptide comprising all or a portion of an AAV capsid comprising a variant VR-I comprising a sequence set forth in any one of SEQ ID NOs: 21-336. Optionally, variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, Hela cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instances, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject, and in particular the liver of a subject, treats a liver-associated disease or condition. In illustrative embodiments, the liver-associated disease or condition is selected from among a urea cycle disorder (UCD; including N-acetylglutamate synthase deficiency (NAGSD), carbamylphosphate synthetase 1 deficiency (CPS1D), ornithine transcarbamylase deficiency (OTCD), argininosuccinate synthetase deficiency (ASSD), argininosuccinate lyase (ASLD), arginase 1 deficiency (ARG1D), citrin or aspartate/glutamate carrier deficiency and the mitochondrial ornithine transporter 1 deficiency causing hyperornithinemia-hyperammonemia-homocitrullinuria syndrome (HHH syndrome)), organic acidopathy (or organic academia, including methylmalonic acidemia, propionic acidemia, isovaleric acidemia, and maple syrup urine disease), aminoacidopathy, glycogenoses (Types I, III and IV), Wilson's disease, Progressive Familial Intrahepatic Cholestasis, primary hyperoxaluria, complementopathy, coagulopathy (e.g. hemophilia A, hemophilia B, von Willebrand disease (VWD)), Crigler Najjar syndrome, familial hypercholesterolaemia, α-1-antitrypsin deficiency, mitochondria respiratory chain hepatopathy, and citrin deficiency. Those skilled in the art would readily be able to select an appropriate heterologous coding sequence useful for treating such diseases. In some examples, the heterologous coding sequence comprises all or a part of a gene that is associated with the disease, such as all or a part of a gene set forth in Table 3. Introduction of such a sequence to the liver can be used for gene replacement or gene editing/correction, e.g. using CRISPR-Cas9. In particular examples, the heterologous coding sequence encodes a protein encoded by a gene that is associated with the disease, such as a gene set forth in Table 3.

TABLE 3

| Exemplary liver-associated diseases | Exemplary associated genes |
|---|---|
| Urea cycle disorders (UCDs) | OTC, ASS, CPS1, ASL, ARG1 |
| Organic acidopathies | PCCA, PCCB, MMUT |
| Aminoacidopathies | PAH, FAH |
| Glycogenoses (Types I, III and IV) | SLC37A4 |
| Wilson's Disease | ATP7B |
| Progressive Familial | ABCB4, ABCB11, ATP8B1 |
| Intrahepatic Cholestasis | |
| Primary Hyperoxaluria | AGXT |
| Complementopathies | CFH, CFI |
| Coagulopathies | F8, F9, VWF |
| Crigler Najjar syndrome | UGT1A1 |
| Familial Hypercholesterolaemia | LDLR |
| α-1-antitrypsin Deficiency | SERPINA1 |
| Mitochondria Respiratory | POLG |
| Chain Hepatopathies | |
| Citrin Deficiency | SLC25A13 |

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

The vectors of the present disclosure can comprise promoters. In instances where the vector is a nucleic acid vector comprising nucleic acid encoding the capsid polypeptide, the promoter may facilitate expression of the nucleic acid encoding the capsid polypeptide. In instances where the vector is an AAV vector, the promoter may facilitate expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In some embodiments, tissue specific promoters are used. Non-limiting examples of such promoters include the liver-specific thyroxin binding globulin (TBG) promoter, insulin promoter, glucagon promoter, somatostatin promoter, pancreatic polypeptide (PPY) promoter, synapsin-1 (Syn) promoter, creatine kinase (MCK) promoter, mammalian desmin (DES) promoter, a α-myosin heavy chain (a-MHC) promoter, a cardiac Troponin T (cTnT) promoter, beta-actin promoter, and hepatitis B virus core promoter. The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a nucleic acid molecule or vector of the present disclosure. In some instances, the host cells are used to amplify, replicate, package and/or purify a polynucleotide or vector. In other examples, the host cells are used to express a heterologous sequence, such as one packaged within an AAV vector. Exemplary host cells include prokaryotic and eukaryotic cells. In some instances, the host cell is a mammalian host cell. It is well within the skill of a skilled artisan to select an appropriate host cell for the expression, amplification, replication, packaging and/or purification of a polynucleotide, vector or rAAV virion of the present disclosure. Exemplary mammalian host cells include, but are not limited to, HEK293 cells, Hela cells, Vero cells, HuH-7 cells, and HepG2 cells. In particular examples, the host cell is a hepatocyte or cell-line derived from a hepatocyte.

### Compositions

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

### Methods

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a hepatocyte (e.g. a human hepatocyte).

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intramuscular, intravaginal, intravenous, intraperitoneal, subcutaneous, epicutaneous, intradermal, rectal, intraocular, pulmonary, intracranial, intraosseous, oral, buccal, or nasal routes. The AAV vector can be administrated as a single dose or multiple doses, and at varying intervals.

Also provided are methods for producing an AAV vector described above and herein, i.e. one comprising a capsid polypeptide of the present disclosure. Such methods comprise culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid polypeptide of the present disclosure, wherein the capsid encapsidates the heterologous coding sequence.

In further aspects, provided are methods for enhancing the in vivo human hepatocyte transduction efficiency of an AAV vector by modifying the VR-I of a reference capsid polypeptide such that the modified VR-I comprises the sequence set forth in any one of SEQ ID NOs: 21-336. Optionally, variant VR-I comprises a sequence set forth in any one of SEQ ID NOs: 253, 251, 196, 274, 319, 288, 194, 231, 193, 266, 207, 273, 243, 286, 209, 256, 310, 220, 198, 283, 275, 223, 212, 328, 254, 67, 157, 129, 64, 117, 166, 45, 35, 164 or 32.

Thus, provided are methods for producing a modified AAV vector that exhibits enhanced transduction efficacy, where the methods include the steps of modifying the sequence of a reference capsid polypeptide (i.e. replacing and/or inserting one or more amino acids) at one or more of positions 262, 263, 264, 265, 266, 267, 268, 269, 270 and 271, with numbering relative to SEQ ID NO:5, to thereby produce a modified capsid polypeptide that comprises a sequence set forth in any one of SEQ ID NOs: 21-336 at positions 262-271, with numbering relative to SEQ ID NO:5.

It will be understood that any modification or combination of modifications, e.g. amino acid replacement or substitution, amino acid deletion and/or amino acid insertion, will result in a change of amino acid sequence in the modified capsid polypeptide compared to the reference capsid polypeptide. Thus, for example, reference to modification does not include within its scope amino acid substitutions where one amino acid residue is substituted with the same amino acid residue, or modifications when an amino acid deletion is accompanied by an insertion of that deleted amino acid, such that there is no difference in the amino acid sequence of the modified capsid polypeptide compared to the reference capsid polypeptide sequence, i.e. the amino acid sequence of the modified capsid polypeptide cannot be the same as (or must be different to) the amino acid sequence of the reference capsid polypeptide sequence.

Typically, the methods include an initial step of first identifying a reference capsid polypeptide for transducing human hepatocytes in vivo. The reference capsid polypeptide may be any AAV polypeptide, such as an AAV1, AAV2, AAV3, AAV3B, AAV4, AAV5, AAV6, AAV7, AAV8, AAV9, AAV10, AAV11, AAV12 or AAV13 capsid polypeptide, or a synthetic or chimeric capsid polypeptide. In illustrative embodiments, the reference polypeptide comprises at least or about 85%, 86%, 87%, 88%, 89%, 90%, 91%, 92%, 93%, 94%, 95%, 96%, 97%, 98%, or 99% sequence identity to the sequence set forth in SEQ ID NO:2 or 4. Reference capsid polypeptides include those comprising all or a portion of the VP1 protein, VP2 protein or VP3 protein. Thus, in some embodiments, the reference capsid polypeptide comprises all or a portion of a VP1 protein having at least or about 85%, 86%, 87%, 88%, 89%, 90%, 91%, 92%, 93%, 94%, 95%, 96%, 97%, 98%, or 99% sequence identity to the sequence set forth in SEQ ID NO: 2 or 4; all or a portion of a VP2 protein having at least or about 85%, 86%, 87%, 88%, 89%, 90%, 91%, 92%, 93%, 94%, 95%, 96%, 97%, 98%, or 99% sequence identity to the VP2 protein set forth as amino acids 138-736 of SEQ ID NO:2 or amino acids 138-735 of SEQ ID NO:4; and all or a portion of a VP3 protein having at least or about 85%, 86%, 87%, 88%, 89%, 90%, 91%, 92%, 93%, 94%, 95%, 96%, 97%, 98%, or 99% sequence identity to the VP3 protein set forth as amino acids 203-736 of SEQ ID NO:2 or 204-735 of SEQ ID NO:4.

Methods for modifying the sequence of a reference capsid polypeptide or polynucleotide so as to produce a modified capsid polypeptide or polynucleotide are well known in the art, and any such method can be utilised so as to perform the methods of the present disclosure. For example, the modification of the sequence of the reference capsid polynucleotide to produce a modified capsid polynucleotide can be performed using any method known in the art, including recombinant and synthetic methods, performed (either in part or in whole) in silico and/or in vitro. In a particular example, the modification of the sequence is performed in silico, followed by de novo synthesis of the modified capsid polynucleotide having the modified sequence (e.g. by gene synthesis methods such as those involving the chemical synthesis of overlapping oligonucleotides following by gene assembly).

The modified capsid polynucleotides may be contained in nucleic acid vector, such as a plasmid, for subsequent expression, replication, amplification and/or manipulation. Vectors suitable for use in bacterial, insect and mammalian cells are widely described and well-known in the art. Those skilled in the art would appreciate that the vectors may also contain additional sequences and elements useful for the replication of the vector in prokaryotic and/or eukaryotic cells, selection of the vector and the expression of a heterologous sequence in a variety of host cells. For example, the vectors can include a prokaryotic replicon, which is a sequence having the ability to direct autonomous replication and maintenance of the vector extrachromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors can be constructed using known techniques, including, without limitation, the standard techniques of restriction endonuclease digestion, ligation, transformation, plasmid purification, in vitro or chemical synthesis of DNA, and DNA sequencing. The vectors comprising a modified capsid polynucleotide may be introduced into a host cell using any method known in the art.

Following modification, the modified capsid are then vectorised. Methods for vectorising a capsid polypeptide are well known in the art and non-limiting examples are described above.

The AAV vector produced by these methods typically has a transduction efficiency that is enhanced compared to a reference AAV vector having a capsid comprising the reference capsid polypeptide. The level of transduction efficiency can be enhanced by at least or about, 10%, 20%, 30%, 40%, 50%, 60%, 70%, 80%, 90%, 100%, 200%, 300%, 400%, 500%, 600%, 700%, 800%, 900% 1000%, or more, e.g. the transduction efficiency of the AAV vector can be at least or about 2×, 3×, 4×, 5×, 6×, 7×, 8×, 9×, 10×, 12×, 13×, 14×, 15×, 16×, 17×, 18×, 19×, 20×, 30×, 40×, 50×, 60×, 70×, 80×, 90×, 100× or more compared to the transduction efficiency from an unmodified AAV vector (i.e. an AAV vector comprising the reference capsid) in vivo. In some examples, this is assessed in an in vivo system that utilises human hepatocytes, such as a small animal (e.g. a mouse) with a chimeric liver comprising human hepatocytes (e.g. the hFRG mouse).

Thus, also provided are AAV vectors produced by the methods of the present disclosure.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Generation of Library—General Methods

DNA and RNA Isolation and cDNA Synthesis

Isolation of DNA and RNA and cDNA synthesis was performed as described in detail before (Cabanes-Creus, 2020, Sci Transl Med 12) without modifications. Briefly, DNA was extracted using a standard phenol: chloroform protocol and RNA with the Direct-Zol kit (Zymogen Cat #R2062).

### AAV Vector Packaging and Viral Production

AAV constructs were packaged into AAV capsids using HEK293 cells and a helper-virus-free system, as described previously (Xiao et al. 1998, J Virol 72, 2224-2232). Genomes were packaged in capsid variants using packaging plasmid constructs harbouring rep genes from AAV2 and a specific cap. All vectors were purified using iodixanol gradient ultracentrifugation, as previously described (Khan et al. 2011, Nat Protoc 6, 482-501).

### Mouse Studies

All animal experimental procedures and care were approved by the joint Children's Medical Research Institute (CMRI) and The Children's Hospital at Westmead Animal Care and Ethics Committee. Fah^{-/-}Rag2^{-/-}Il2rg^{-/-} (FRG) mice (Azuma et al., 2007, Nat Biotechnol 25, 903-910) were bred, housed, engrafted, and monitored as recently described (Cabanes-Creuset al., 2020, Mol Ther Methods Clin Dev 17, 1139-1154). Levels of human cell engraftment were estimated by measuring the presence of human albumin in peripheral blood, using the human albumin ELISA quantitation kit (Bethyl Laboratories, catalogue no. E80-129). To evaluate the AAV transduction potential, mice were placed on 10% NTBC and were maintained in this condition until harvest. Mice were randomly assigned to experiments and transduced via intravenous injection (lateral tail vein) with the indicated vector doses. In some instances, mice were first passively immunized with an injection of 5 mg soluble IVIg. Mice were euthanised by CO₂ inhalation 1 week after transduction. To obtain murine and human single-cell suspensions from xenografted murine livers, the same collagenase perfusion procedure as recently described was followed (Cabanes-Creus et al., 2020, Sci Transl Med 12). For all experiments, cells were labelled with phycoerythrin (PE)-conjugated anti-human-HLA-ABC (clone W6/32, Invitrogen 12-9983-42; 1:20), biotin-conjugated anti-mouse-H-2Kb (clone AF6-88.5, BD Pharmigen 553568; 1:100), and allophycocyanin (APC)-conjugated-streptavidin (eBioscience 17-4317-82; 1:500). GFP-positive-labelled samples were sorted to a minimal 95% purity using a BD Influx Cell sorter. Flow cytometry was performed in the Flow Cytometry Facility, Westmead Institute for Medical Research (WIMR), Westmead, NSW, Australia. The data were analysed using FlowJo 7.6.1 (FlowJo LLC).

### Liver Explants

Liver explants were prepared using normothermic liver perfusion and anatomical split. The perfusion system was modified from a commercial liver perfusion machine (Liver assist, Organ Assist, Groningen, Netherlands). A dialysis membrane, two long-term oxygenators and a gas blender are added to the system to extent organ viability. The perfusate contains red blood cells, two units of fresh frozen plasma and albumin, and it is maintained at 36° C. The graft receives infusions of taurocholic acid (7.7 mg/hr), methylprednisolone (50 mg/24 hr), parenteral nutrition (20 mL/hr), and titratable insulin and glucagon. 20 hours after perfusion, the whole liver was anatomically split, given that all viability requirements were achieved. These were lactate<2.5 mmol/L and two or more of the following: bile production, arterial pH>7.3, evidence of glucose metabolism, homogeneous perfusion, and bile pH>7.4. The AAV library was injected in the portal vein of the left lobe. Two days post-injection, core biopsies were taken, and DNA and RNA were extracted for AAV isolation.

### Sequencing

The region surrounding the synonymous codon barcode and the VR-I library was analysed with next-generation sequencing, with the NGS-F and NGS-R primers presented below in Table 22. NGS analysis was performed on the plasmid library, vector library, and transduced hepatocytes (DNA and cDNA). NGS library preparations and sequencing using a 2×150 paired-end configurations were performed by Genewiz (Suzhou, China) using an Illumina MiSeq instrument. A workflow was written in Snakemake (5.6) (Koster et al., 2018, Bioinformatics 34, 3600) to process reads and count barcodes. Paired reads were merged using BBMerge and then filtered for reads of the expected length in a second pass through BBDuk, both from BBTools 38.68 (https://sourceforge.net/projects/bbmap/). The merged, filtered fastq files were passed to a Python (3.7) script that identified barcodes corresponding to AAV variants. NGS reads from the DNA and cDNA populations were normalized to the reads from the pre-injection mix.

Analysis of Library Variants in hFRG Mice

To assess the performance of variants following injection of an hFRG mouse with a VR-I library, a series of calculations were made that reflected the ability of the variants to enter hepatocytes (analysis of DNA; “physical transduction”), and to express following entry (analysis of cDNA; “functional transduction”).

For each variant, the following scores were calculated in triplicate:

$$\begin{matrix}Entry Index = log 2 [ \% Reads at DNA ] / [ \% reads at Viral Library ] \\ Expression Index = log 2 [ \% Reads at cDNA ] / [ \% reads at Viral Library ]\end{matrix}$$

To rank the variants, a strategy that aimed to maximize consistent performance (i.e. similarity of performance of a given variant across the triplicates independently of synonymous codon) was utilised. For that fitness scores were created:

$$\begin{matrix}Entry Fitness = Entry Index 01 + Entry Index 02 + Entry Index 03 \\ Expression Fitness = Expression Index 01 + Expression Index 02 + Expression Index 03\end{matrix}$$

Similarly, variants that performed differently with different synonymous codons were penalised. To do this, the standard deviation of both entry and expression index was determined before a correcting formula that penalized variants with high standard deviations was applied:

$$Corrected Fitness = Fitness - ( 0.5 \times {Deviation}^{2} )$$

The total score then was then calculated as the sum of Corrected Entry Fitness and Corrected Expression Fitness.

### Example 2. Generation of VR-I Library

VR-I libraries were built on the LK03-R594E+D598H capsid backbone (also referred to as LK03-REDH; capsid sequence set forth in SEQ ID NO:1 (nt) and SEQ ID NO:2 (aa)) and on the C11.11 capsid backbone (capsid sequence set forth in SEQ ID NO:3 (nt) and SEQ ID NO:4 (aa)). LK03-REDH is a capsid based on LK03 (which is itself an AAV3B-like capsid polypeptide), but further comprising R594E and D598H mutations. Given the homology surrounding the VR-I, the process to clone the different libraries was the same.

The sequences of the VR-I from AAV1, AAV2, AAV3b, AAV7, AAV8, AAV9 and AAV10 capsids were aligned (FIG. 1). Position 262 in AAV2 corresponds to position 262 in AAV1, AAV3b and AAV9 capsids (and LK03-REDH), and position 263 in AAV7, AAV8 and AAV10 capsids (and C11.11). AAV1 and AAV7 capsids harbour one amino acid insertion within the VR-I and AAV8, AAV9, and AAV10 capsids harbour two amino acid insertions within VR-I, when compared to the AAV2 capsid. For each of positions 262-271 (with numbering relative to the AAV2 capsid set forth in SEQ ID NO:5), the amino acids present in any of the AAVs were listed, as shown in Table 4. The library variants were therefore constructed to contain either 10, 11 or 12 amino acids in this region, with any combination of the residues (or deletions) shown in Table 4 (with numbering relative to the AAV2 capsid shown in the top row).

TABLE 4
Amino acid residues at positions 262-271 from different serotypes

| | | | | | AAV2 | AAV2 | AAV2 | AAV2 | AAV2 | AAV2 | AAV2 |
|---|---|---|---|---|---|---|---|---|---|---|---|
| AAV2 | | | INS | INS | 265 | 266 | 267 | 268 | 269 | 270 | 271 |
| 262 | 263 | 264 | 265 | 266 | 267 | 268 | 269 | 270 | 271 | 272 | 273 |
| S | Q | S | A | G | G | A | S | N | D | N | T |
| N | S | T | S | | | S | T | | | | A |
| | A | | T | | | | | | | | H |
| | G | | | | | | | | | | |
| | E | | | | | | | | | | |

For each position, the ‘AA-Calculator’ tool (http://guinevere.otago.ac.nz/cgi-bin/aef/AA-Calculator.pl) (Firth, 2008) was used to find optimal codons to encode selected amino acids. The criteria for choosing the degenerate codon was the following: 1) avoided stop-codons; 2) contained all the naturally existing amino acids in AAV; 3) lower number of codified codons. If #2 was not possible due to limitations of the genetic code, then a degenerate codon was chosen following: 4) lower number of codified amino acids; and 5) lower number of codified codons. For example, for position 262, the naturally occurring amino acids are Asn (N) and Ser (S). The trinucleotide ART or ARC would be chosen before ARY, given that the latter codifies for 4 codons and thus would increase the library complexity at the codon level. In some instances, for example for position 263, it was not possible to find a degenerate codon that codifies only for Q,S,A,G,E. Thus, other amino acids that do not exist in the selected wild-type variants, were included, as exemplified below. The trinucleotide VVW was chosen, since it does not encode for stop codons and minimized the number of amino acids to 12 and the number of codons to 18. Table 5 summarizes the chosen degenerate codons (in italics) for each position, and amino acids (AA) for which those degenerate codons codify. Amino acids not present in any of the chosen wild-type AAVs but included in the library are also shown (in brackets).

TABLE 5
Codons and amino acid residues for inclusion at positions 262-271

| | AAV2 | AAV2 | AAV2 | | | AAV2 | AAV2 | AAV2 | AAV2 | AAV2 | AAV2 | AAV2 |
|---|---|---|---|---|---|---|---|---|---|---|---|---|
| | 262 | 263 | 264 | INS | INS | 265 | 266 | 267 | 268 | 269 | 270 | 271 |
| | 262 | 263 | 264 | 265 | 266 | 267 | 268 | 269 | 270 | 271 | 272 | 273 |
| Codon | ARC | VVW | WCW | RVC | GGA | GGA | KCT | WCW | AAC | GAC | AAC | VMC |
| AA | S | Q | S | A | G | G | A | S | N | D | N | T |
| | N | S | T | S | | | S | T | | | | A |
| | | A | | T | | | | | | | | H |
| | | G | | | | | | | | | | |
| | | E | | | | | | | | | | |
| | | (D) | | (N) | | | | | | | | (P) |
| | | (H) | | (D) | | | | | | | | (D) |
| | | (K) | | (G) | | | | | | | | (N) |
| | | (N) | | | | | | | | | | |
| | | (P) | | | | | | | | | | |
| | | (T) | | | | | | | | | | |
| | | (R) | | | | | | | | | | |

Given that the region surrounding VR-I is common between the C11.11 and LK03-REDH capsids, the same cloning strategy was used for each. The capsid region was cloned into a cloning plasmid with Trimethoprim resistance, with Swa-I and Nsi-I restriction sites surrounding the capsid insert. A stop codon was cloned at Q263 (numbering relative to the LK03-REDH capsid set forth in SEQ ID NO:2) so as to reduce packaging of the background plasmid coding. A PCR-based strategy was then utilized for cloning, with three forward primers having a common region aligning to the capsid region downstream of the VR-I region and degenerate codons matching the table described above. Each forward primer (Fwd 10aa, Fwd 11aa and Fwd 12aa) was used in a different PCR reaction with a common reverse primer (Rev common 1) (see Table 22 for sequences). Each forward primer ‘fixes’ the Q263_Stop-Codon. Moreover, the linear PCR product harbours compatible, complementary ends for Gibson Assembly and thus can be self-annealed generating a functional plasmid. The PCRs were run independently, and the products were purified and maintained separately to minimize molar differences among individual variants. 11 aa/12 aa PCR products contain 6 times the number of variants of the 10 aa product. To account for these differences, the PCR products were mixed at 1:6:6 molar ratio (10/11/12 aa) prior to proceeding with Gibson Assembly. The reactions were carried out following manufacturer's instructions, mixing a total of 1 pmol.

In order to reduce the noise associated with library selection, a novel strategy to generate intra-animal replicates was developed. With that purpose, and following the exact same protocol as described above, two additional independent VR-I libraries for each capsid backbone were cloned by changing only the reverse primer. Rev common 2 and Rev common 3 (see Table 22) were generated. Amino acids TYNN are present at positions 250-253 of LK03-REDH and C11.11. Synonymous codons maintaining TYNN but allowing ‘barcoding’ at the DNA-level were utilised in Rev common 2 and Rev common 3, which covered this region.

The three libraries were then mixed at the packaging step and analysed by 150-PE Illumina Next-Generation Sequencing of a single fragment, with PCR primers surrounding both the synonymous codons barcodes and the library itself.

The libraries contain 14976 variants, consisting of all the possible amino acid combinations present in Table 4. Compared to the C11.11 and LK03-REDH capsids, which contain the 10 aa VR-I region from AAV2/3b, some variants in the library include a 1 amino acid or 2 amino acid insertion after position 264 (with numbering relative to the AAV2 capsid). Consequently, the library consists of variants having a 10, 11 or 12 amino acid VR-I.

The libraries were cloned into the Functional Transduction Platform (harbouring the Liver-Specific Promoter (LSP)) and packaged, as described previously (Cabanes-Creus et al., 2020, Methods Clin Dev, 17:1139-1154; see also PCT/AU2019/051133, the disclosure of which is incorporated herein by reference).

### Example 3. Identification of Variants with Improved Transduction

The libraries were injected into highly engrafted hFRG mice (Example 1). Specifically, the AAV-LK03-REDH and AAVC11.11 libraries were screened independently in two highly humanized FRG mice, and one highly humanized FRG mouse passively immunized with 5 mg of IVIg 24 hours prior to AAV injection. DNA and cDNA were extracted from sorted human hepatocytes one week after injection.

The libraries were also screened jointly in the left-lobe of a human liver explant in a perfusion system (see Example 1). DNA and cDNA were recovered two days post library injection. To distinguish between AAV-LK03-REDH and AAVC11.11, the libraries that went into the human explant were further barcoded as follows, at I240 (LK03-REDH numbering):

| | Amino Acids | Syn-Codon Capsid |
|---|---|---|
| REDH | ITT | ATCACAACA |
| AAVC11.11 | ITT | ATTACTACG |

Primers for NGS from the liver explant were the forward and reverse primer sequences of SEQ ID NO:337 and SEQ ID NO: 20, respectively; see Table 22).

Vector DNA and RNA (cDNA) levels in human hepatocytes isolated from the liver were assessed by NGS (using the forward and reverse primer sequences of SEQ ID NOs: 19 and 20, respectively; see Table 22), and the various Entry and Expression Scores, and Total Scores, were calculated as described above. Variants were then ranked from 1 to 14976 based on their Total Score.

Table 6 provides the top 40 variants from the library based on the AAV-LK03-REDH backbone, screened in an hFRG mouse with no IVIg. Interestingly, the vector containing the wild-type VR-I (SQSGASNDNH; SEQ ID NO:12) from AAV-LK03-REDH ranked at 1932. Importantly, a relatively higher entry score than expression score was observed for this vector, which is consistent with that observed for AAV-LK03-REDH. Table 7 provides the top 40 variants from this library screened in a separate hFRG mouse with no IVIg; Table 8 provides the top 40 variants from this library screened in a hFRG mouse with IVIg; and Table 9 provides the top 40 variants from this library screened in liver explants. Tables 10-13 provides the top 40 variants from the library based on the AAVC11.11 backbone, screened in two separate hFRG mice (no IVIG) and a hFRG mouse with IVIg, and a liver explant, respectively.

TABLE 6
Scores for AAV-LK03-REDH VR-I variants-
hFRG (no IVIg) replicate 1

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | SRSAGGASNDNA | 10.515 | 10.501 | 11.863 | 11.167 | 21.668 |
| 2 | SRSSGGSTNDNA | 17.648 | 12.642 | 18.872 | 6.964 | 19.607 |
| 3 | NRTGGGATNDNA | 12.640 | 12.542 | 6.453 | 6.194 | 18.737 |
| 4 | NTTAGSSNDNP | 11.727 | 8.515 | 16.384 | 9.784 | 18.299 |
| 5 | NRTAGGSTNDNH | 11.633 | 11.267 | 6.123 | 5.773 | 17.041 |
| 6 | SRSSGGSSNDNT | 11.065 | 10.193 | 8.318 | 6.390 | 16.583 |
| 7 | SKSTGASNDNN | 11.273 | 10.531 | 6.890 | 5.953 | 16.484 |
| 8 | SRSSGGASNDNH | 7.790 | 7.481 | 10.274 | 8.909 | 16.390 |
| 9 | NKTGGGASNDNT | 12.136 | 12.118 | 4.524 | 4.246 | 16.364 |
| 10 | NKTNGGATNDNH | 9.204 | 8.983 | 8.973 | 7.263 | 16.247 |
| 11 | NRTTGGATNDNH | 9.025 | 9.018 | 9.074 | 6.476 | 15.494 |
| 12 | NKTGGASNDNT | 11.231 | 10.452 | 10.890 | 4.767 | 15.218 |
| 13 | SKSAGGSSNDNA | 12.151 | 11.719 | 3.664 | 3.168 | 14.887 |
| 14 | NRTSGGASNDNH | 10.248 | 10.166 | 6.685 | 4.456 | 14.621 |
| 15 | SRSTGGASNDNA | 10.731 | 10.279 | 9.287 | 4.240 | 14.519 |
| 16 | SKSTGASNDNA | 10.974 | 10.300 | 4.494 | 4.204 | 14.503 |
| 17 | NKTAGGATNDNT | 12.385 | 11.009 | 3.716 | 3.492 | 14.501 |
| 18 | NRSSGGATNDNA | 11.931 | 11.253 | 4.609 | 2.923 | 14.176 |
| 19 | SRSSGGASNDNA | 10.334 | 10.243 | 6.217 | 3.887 | 14.129 |
| 20 | SKTTGGSSNDNA | 11.778 | 11.718 | 2.646 | 2.334 | 14.052 |
| 21 | SKSAGGASNDNH | 9.720 | 9.480 | 8.265 | 4.523 | 14.003 |
| 22 | SRSTGGASNDNN | 9.428 | 9.370 | 4.934 | 4.216 | 13.585 |
| 23 | SRSTGGSSNDNA | 10.489 | 10.278 | 3.494 | 3.159 | 13.437 |
| 24 | NKTAGGSTNDNH | 10.351 | 10.142 | 3.231 | 3.213 | 13.355 |
| 25 | NRTNGGATNDNT | 8.685 | 8.667 | 4.999 | 4.610 | 13.277 |
| 26 | NRTTGGSTNDNN | 9.852 | 9.444 | 3.765 | 3.723 | 13.167 |
| 27 | NKTTGGATNDNH | 9.293 | 8.541 | 11.013 | 4.583 | 13.124 |
| 28 | NNSGATNDNH | 5.867 | 5.019 | 11.124 | 7.950 | 12.969 |
| 29 | NHSNGATNDNN | 4.958 | 4.775 | 8.814 | 8.163 | 12.938 |
| 30 | SKTSGSSNDNH | 11.562 | 10.395 | 4.247 | 2.495 | 12.890 |
| 31 | SRSAGGSSNDNT | 10.610 | 9.897 | 3.787 | 2.979 | 12.876 |
| 32 | NGTAGSSNDNN | 9.704 | 6.575 | 12.182 | 6.295 | 12.870 |
| 33 | NRTSGGASNDNA | 9.184 | 9.118 | 3.676 | 3.668 | 12.786 |
| 34 | SSSSGGASNDNN | 5.998 | 5.189 | 10.122 | 7.561 | 12.749 |
| 35 | SRSSGGSSNDNA | 9.714 | 9.295 | 3.439 | 3.434 | 12.728 |
| 36 | NKTNGGASNDNN | 7.418 | 7.394 | 8.247 | 5.302 | 12.696 |
| 37 | NKTGGGASNDNH | 8.646 | 8.458 | 5.344 | 4.180 | 12.638 |
| 38 | SRTTGGSSNDNH | 10.204 | 9.522 | 5.485 | 3.077 | 12.599 |
| 39 | SRTAGGATNDNN | 9.083 | 7.427 | 11.728 | 5.163 | 12.590 |
| 40 | SRSGGGSSNDNT | 9.344 | 9.314 | 5.374 | 3.186 | 12.500 |
| 1928 | SQSGASNDNH | 2.731 | 2.410 | −4.359 | −4.969 | −2.559 |

TABLE 7
Scores for AAV-LK03-REDH VR-I variants-
hFRG (no IVIg) replicate 2

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | NHTSGSSNDNH | 8.194 | 7.937 | 11.165 | 10.795 | 18.731 |
| 2 | SKSTGSTNDNH | 10.431 | 8.487 | 11.960 | 6.788 | 15.275 |
| 3 | NSTGGSSNDNA | 9.555 | 7.513 | 11.802 | 7.610 | 15.124 |
| 4 | NESGGGASNDNN | 9.612 | 7.401 | 12.808 | 7.088 | 14.489 |
| 5 | NNSSGATNDNN | 8.187 | 5.665 | 12.481 | 8.725 | 14.391 |
| 6 | NRTNGGATNDNH | 5.808 | 5.434 | 8.908 | 7.960 | 13.395 |
| 7 | NRTGGGATNDNH | 6.476 | 5.778 | 8.419 | 4.702 | 10.480 |
| 8 | NASTGGATNDNN | 6.864 | 6.134 | 7.334 | 3.966 | 10.100 |
| 9 | SDTNGATNDNN | 5.337 | 5.047 | 7.671 | 4.958 | 10.004 |
| 10 | SASGSSNDNH | 6.043 | 5.706 | 7.194 | 3.929 | 9.636 |
| 11 | NDSTGGATNDNN | 9.948 | 6.507 | 10.925 | 2.949 | 9.456 |
| 12 | NATTGGASNDNA | 7.800 | 5.477 | 9.985 | 3.739 | 9.216 |
| 13 | SKTNGSSNDNH | 7.790 | 5.946 | 10.034 | 2.971 | 8.917 |
| 14 | STTSGASNDNA | 5.268 | 5.103 | 4.355 | 3.773 | 8.876 |
| 15 | SHTAGGSSNDNH | 8.474 | 6.546 | 8.498 | 2.005 | 8.551 |
| 16 | STSAGASNDNH | 3.658 | 3.293 | 6.725 | 5.197 | 8.490 |
| 17 | NRSGGATNDNH | 8.210 | 4.336 | 11.618 | 4.095 | 8.431 |
| 18 | NTSGGSSNDNN | 7.000 | 5.083 | 8.252 | 3.073 | 8.156 |
| 19 | NHSAGGASNDNA | 8.067 | 7.028 | 6.567 | 1.037 | 8.065 |
| 20 | SETSGGSSNDNH | 12.075 | 5.198 | 13.954 | 2.659 | 7.856 |
| 21 | SASNGGSSNDNA | 8.148 | 5.685 | 8.612 | 2.103 | 7.788 |
| 22 | SDTGGGASNDNT | 8.077 | 6.049 | 7.881 | 1.661 | 7.710 |
| 23 | SSSGGGATNDNA | 7.510 | 5.231 | 8.422 | 2.465 | 7.696 |
| 24 | SNSAGGSSNDNA | 5.148 | 4.990 | 3.671 | 2.700 | 7.690 |
| 25 | SSTNGGSTNDNH | 7.053 | 6.852 | 1.447 | 0.813 | 7.665 |
| 26 | SGTGGGASNDNA | 4.287 | 3.856 | 6.019 | 3.795 | 7.651 |
| 27 | SGTTGGATNDNN | 7.773 | 5.976 | 7.544 | 1.661 | 7.637 |
| 28 | SGTSGGASNDNA | 3.362 | 3.230 | 4.762 | 4.373 | 7.604 |
| 29 | NDSSGSSNDNT | 8.342 | 4.224 | 10.542 | 3.336 | 7.560 |
| 30 | NKSTGASNDNN | 5.804 | 4.068 | 7.784 | 3.467 | 7.535 |
| 31 | NQTGGSSNDNA | 3.851 | 3.846 | 4.569 | 3.669 | 7.516 |
| 32 | SNSGATNDNH | 6.751 | 4.652 | 9.287 | 2.465 | 7.117 |
| 33 | SHSSGGSSNDNH | 8.756 | 5.851 | 9.127 | 1.088 | 6.940 |
| 34 | SQTGGGASNDNH | 5.617 | 5.018 | 6.989 | 1.821 | 6.838 |
| 35 | SKTSGSSNDNN | 6.118 | 5.358 | 4.420 | 1.342 | 6.700 |
| 36 | SRSSGGASNDNT | 5.042 | 4.287 | 6.437 | 2.275 | 6.562 |
| 37 | NRSTGASNDNN | 8.720 | 5.322 | 11.355 | 1.200 | 6.522 |
| 38 | NGSNGGASNDNN | 4.879 | 3.996 | 6.835 | 2.491 | 6.486 |
| 39 | SRTNGGSTNDNA | 6.185 | 5.878 | 0.961 | 0.533 | 6.411 |
| 40 | SSSGGSTNDNA | 3.914 | 2.494 | 6.492 | 3.711 | 6.204 |
| 8811 | SQSGASNDNH | −4.641 | −5.501 | −11.815 | −12.363 | −17.864 |

TABLE 8
Scores for AAV-LK03-REDH VR-I variants-
hFRG + IVIg

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | SGTNGGSTNDNT | 10.025 | 9.553 | 9.549 | 2.644 | 12.197 |
| 2 | SRSDGGSTNDNN | 8.326 | 8.070 | 3.536 | 3.371 | 11.441 |
| 3 | STTSGASNDNA | 7.677 | 6.840 | 7.088 | 3.090 | 9.929 |
| 4 | SGSNGGASNDNN | 7.196 | 5.831 | 8.703 | 3.385 | 9.216 |
| 5 | NRSNGGSSNDNH | 12.710 | 7.960 | 12.536 | 0.754 | 8.714 |
| 6 | SKTNGGASNDNH | 6.957 | 5.694 | 8.077 | 2.898 | 8.592 |
| 7 | SRSSGGATNDNA | 9.708 | 6.707 | 9.623 | 1.854 | 8.561 |
| 8 | SSSTGGATNDNA | 6.020 | 5.625 | 2.947 | 2.717 | 8.342 |
| 9 | NDTGGGATNDNP | 6.171 | 6.171 | 2.051 | 1.855 | 8.026 |
| 10 | SGSGGGSTNDNA | 5.300 | 4.758 | 6.210 | 3.260 | 8.018 |
| 11 | SSSGGGSTNDNA | 9.995 | 7.547 | 8.365 | 0.340 | 7.886 |
| 12 | SDTSGATNDNA | 6.991 | 1.888 | 11.709 | 5.918 | 7.806 |
| 13 | NASTGGATNDNP | 4.746 | 4.444 | 3.314 | 3.309 | 7.752 |
| 14 | NKTSGGSSNDNA | 5.485 | 5.414 | 4.498 | 2.196 | 7.610 |
| 15 | NATGGGATNDNP | 7.586 | 6.932 | 0.977 | 0.649 | 7.581 |
| 16 | SKTNGGSTNDNA | 8.654 | 7.260 | 1.899 | 0.319 | 7.579 |
| 17 | SGTNGGSSNDNT | 7.410 | 6.711 | 1.194 | 0.379 | 7.091 |
| 18 | NTTAGGSTNDNN | 9.395 | 7.181 | 7.717 | −0.118 | 7.063 |
| 19 | SQTGGGASNDNT | 5.947 | 5.746 | 4.022 | 1.170 | 6.917 |
| 20 | NTTTGATNDNT | 5.373 | 5.255 | 1.638 | 1.631 | 6.885 |
| 21 | SHTAGSSNDNA | 6.692 | 6.509 | 1.384 | 0.358 | 6.867 |
| 22 | SKSSGSSNDNA | 4.999 | 4.843 | 2.314 | 1.907 | 6.751 |
| 23 | SSSDGGSTNDNA | 4.339 | 3.836 | 2.761 | 2.761 | 6.597 |
| 24 | SNTTGGSTNDNH | 7.401 | 6.312 | 0.882 | 0.193 | 6.505 |
| 25 | NQTGGGATNDNT | 7.745 | 7.114 | 5.037 | −0.680 | 6.434 |
| 26 | NRSDGGSTNDNN | 4.050 | 3.764 | 2.636 | 2.636 | 6.400 |
| 27 | SGTGGGASNDNH | 4.627 | 3.995 | 6.339 | 2.353 | 6.349 |
| 28 | SNSSGGSTNDNA | 7.245 | 7.155 | −0.686 | −0.836 | 6.319 |
| 29 | NSTNGGATNDNA | 6.606 | 5.110 | 1.998 | 1.095 | 6.206 |
| 30 | NSSSGGATNDNT | 6.479 | 5.062 | 1.466 | 1.014 | 6.076 |
| 31 | SQSSGGATNDNH | 5.719 | 5.607 | 1.544 | 0.452 | 6.059 |
| 32 | SKSDGGSTNDNH | 4.830 | 4.630 | 1.510 | 1.410 | 6.039 |
| 33 | SNTAGGSSNDNT | 7.079 | 6.625 | −0.271 | −0.607 | 6.018 |
| 34 | NESAGGSSNDNN | 5.339 | 5.198 | 1.861 | 0.812 | 6.011 |
| 35 | NHSGGGATNDNP | 5.315 | 5.287 | 0.904 | 0.697 | 5.984 |
| 36 | SSSSGGATNDNA | 6.126 | 6.007 | 0.113 | −0.044 | 5.963 |
| 37 | SHTDGSTNDNH | 4.548 | 4.547 | 1.371 | 1.320 | 5.867 |
| 38 | SATDGGSTNDNA | 6.455 | 6.394 | −0.397 | −0.544 | 5.850 |
| 39 | SKSSGATNDNT | 6.948 | 6.396 | 0.273 | −0.711 | 5.685 |
| 40 | NRSTGGSTNDNT | 4.744 | 4.550 | 1.337 | 1.088 | 5.638 |
| 5681 | SQSGASNDNH | −3.868 | −3.906 | −7.494 | −7.661 | −11.568 |

TABLE 9
Scores for AAV-LK03-REDH VR-I variants-
liver explant

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | SPTDGGSTNDNN | 21.593 | 15.422 | 22.640 | 8.309 | 23.731 |
| 2 | SESAGGSTNDNN | 11.527 | 9.809 | 5.537 | 2.756 | 12.566 |
| 3 | NSSGGGSTNDNT | 10.330 | 9.230 | 2.992 | 2.026 | 11.255 |
| 4 | NDTSGGATNDNT | 9.112 | 9.024 | 1.969 | 1.351 | 10.375 |
| 5 | NNTGSSNDNH | 7.906 | 7.735 | 2.391 | 2.272 | 10.007 |
| 6 | NKSSGGSTNDNN | 9.035 | 8.314 | 1.844 | 1.607 | 9.921 |
| 7 | STTSGGSTNDND | 7.924 | 7.589 | 3.534 | 2.030 | 9.620 |
| 8 | SHSAGGSTNDNA | 8.481 | 8.341 | 1.259 | 1.221 | 9.562 |
| 9 | SDTAGGSTNDNN | 7.780 | 6.855 | 3.364 | 2.626 | 9.481 |
| 10 | SRTDGSTNDNP | 8.092 | 7.428 | 2.166 | 2.041 | 9.470 |
| 11 | NHSGATNDNP | 7.683 | 7.592 | 2.844 | 1.871 | 9.463 |
| 12 | NSSGGATNDNA | 9.319 | 7.969 | 1.747 | 1.004 | 8.973 |
| 13 | NNSNGSSNDNA | 9.098 | 8.675 | 1.384 | −0.262 | 8.412 |
| 14 | NPSNGGSSNDND | 6.557 | 6.554 | 1.849 | 1.839 | 8.393 |
| 15 | SQTNGGSTNDND | 7.511 | 7.137 | 1.212 | 0.942 | 8.078 |
| 16 | SDSGSSNDNT | 5.132 | 4.690 | 3.103 | 2.945 | 7.635 |
| 17 | NESAGATNDNN | 7.515 | 6.888 | 0.879 | 0.612 | 7.500 |
| 18 | NPSAGGSSNDNN | 7.321 | 6.064 | 1.754 | 1.313 | 7.377 |
| 19 | SSTGATNDNT | 8.043 | 5.196 | 2.976 | 2.079 | 7.275 |
| 20 | SESNGGSSNDNT | 7.936 | 6.645 | 1.949 | 0.623 | 7.269 |
| 21 | SDTNGSTNDNA | 5.057 | 5.003 | 2.259 | 2.165 | 7.168 |
| 22 | NPSGASNDNT | 5.458 | 5.386 | 1.391 | 1.324 | 6.710 |
| 23 | STSGGGSTNDNP | 4.406 | 4.129 | 2.522 | 2.500 | 6.629 |
| 24 | NHSTGGATNDND | 6.689 | 5.444 | 0.489 | 0.402 | 5.846 |
| 25 | NDSGGATNDNT | 4.871 | 4.868 | 0.890 | 0.761 | 5.629 |
| 26 | SHTDGGSSNDNA | 6.178 | 6.014 | −0.200 | −0.559 | 5.455 |
| 27 | SKSSGATNDNA | 6.371 | 5.978 | 0.674 | −0.533 | 5.445 |
| 28 | SKTGSTNDND | 7.734 | 7.690 | −2.259 | −2.284 | 5.406 |
| 29 | SQTGGGSSNDNP | 5.993 | 5.081 | 0.391 | 0.272 | 5.354 |
| 30 | SHTSGSSNDNP | 7.010 | 6.615 | −0.139 | −1.327 | 5.287 |
| 31 | NASSGSSNDND | 5.338 | 5.333 | −0.083 | −0.107 | 5.227 |
| 32 | SSTAGGSTNDNH | 6.174 | 6.068 | 0.522 | −0.858 | 5.209 |
| 33 | NGSDGGSTNDND | 5.589 | 5.561 | −0.194 | −0.412 | 5.149 |
| 34 | STSSGGSSNDNP | 5.098 | 4.078 | 1.107 | 0.913 | 4.992 |
| 35 | SNTGGASNDNA | 5.544 | 4.767 | 2.094 | 0.187 | 4.954 |
| 36 | NDTDGASNDNA | 4.390 | 4.390 | 0.508 | 0.426 | 4.817 |
| 37 | SDTTGATNDNN | 5.157 | 5.155 | −0.346 | −0.346 | 4.808 |
| 38 | SPSGSTNDNN | 4.671 | 4.671 | 0.142 | 0.128 | 4.799 |
| 39 | NRSAGATNDNP | 7.588 | 7.322 | −2.373 | −2.537 | 4.785 |
| 40 | NTSGATNDNT | 6.304 | 5.288 | 1.949 | −0.504 | 4.783 |
| 2792 | SQSGASNDNH | 0.562 | 0.379 | −9.158 | −9.168 | −8.789 |

TABLE 10
Scores for AAVC11.11 VR-I variants-hFRG (no IVIg) replicate 1

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | NRTGGSTNDNA | 12.004 | 11.469 | 7.595 | 7.386 | 18.855 |
| 2 | SETGGATNDNT | 13.569 | 8.966 | 16.685 | 7.118 | 16.084 |
| 3 | SATNGGSSNDNA | 9.951 | 7.429 | 10.295 | 6.482 | 13.911 |
| 4 | SKTDGSSNDNT | 11.317 | 10.259 | 3.491 | 3.047 | 13.306 |
| 5 | NGTSGSTNDNN | 10.525 | 10.306 | 3.249 | 2.965 | 13.270 |
| 6 | NNSSGGSSNDNA | 11.472 | 6.824 | 5.781 | 5.668 | 12.492 |
| 7 | NQSTGGSSNDNT | 8.372 | 8.370 | 4.290 | 3.064 | 11.434 |
| 8 | NQSNGSTNDNA | 9.781 | 7.527 | 3.996 | 3.873 | 11.400 |
| 9 | SSTTGSTNDNA | 9.701 | 7.482 | 4.485 | 3.506 | 10.987 |
| 10 | NDSGGSTNDNT | 8.156 | 8.083 | 3.030 | 2.823 | 10.906 |
| 11 | NETSGGSSNDNA | 8.971 | 8.505 | 2.225 | 2.032 | 10.537 |
| 12 | NSTDGATNDNP | 8.086 | 7.642 | 3.413 | 2.822 | 10.464 |
| 13 | NRSSGGSTNDNH | 9.889 | 9.192 | 8.059 | 1.184 | 10.376 |
| 14 | SKTGGATNDNA | 7.907 | 7.897 | 2.845 | 2.346 | 10.243 |
| 15 | NKSTGGATNDNT | 9.646 | 7.005 | 4.100 | 3.022 | 10.027 |
| 16 | NSSTGSSNDNA | 8.938 | 8.925 | 1.170 | 1.002 | 9.927 |
| 17 | NRSAGGSSNDNH | 8.614 | 7.935 | 2.034 | 1.862 | 9.797 |
| 18 | NDTSGGSSNDNA | 9.524 | 8.157 | 3.775 | 1.419 | 9.576 |
| 19 | SKSTGSSNDNA | 8.966 | 7.538 | 2.042 | 2.035 | 9.573 |
| 20 | SHTTGATNDNT | 7.406 | 7.382 | 2.058 | 1.960 | 9.342 |
| 21 | NNSNGATNDNT | 9.839 | 8.727 | 1.381 | 0.561 | 9.288 |
| 22 | NKSAGGATNDNN | 8.419 | 8.268 | 1.047 | 0.762 | 9.030 |
| 23 | NGTDGGSTNDNN | 7.534 | 7.500 | 2.111 | 1.180 | 8.680 |
| 24 | NKSTGSTNDNH | 10.332 | 5.431 | 6.115 | 3.053 | 8.484 |
| 25 | STTTGSTNDNT | 9.380 | 5.314 | 5.131 | 2.956 | 8.270 |
| 26 | NRTTGGATNDNA | 7.907 | 7.483 | 0.734 | 0.679 | 8.162 |
| 27 | NGTNGGSTNDNH | 7.370 | 7.367 | 0.705 | 0.689 | 8.057 |
| 28 | SKTTGGASNDNT | 8.911 | 7.297 | 2.000 | 0.679 | 7.976 |
| 29 | NNTAGGSSNDNA | 8.878 | 5.636 | 3.338 | 2.336 | 7.971 |
| 30 | SSTNGGSSNDNH | 6.640 | 5.892 | 2.226 | 2.038 | 7.930 |
| 31 | SNTSGGATNDNA | 7.902 | 7.863 | 0.938 | 0.015 | 7.878 |
| 32 | STTSGATNDNT | 7.456 | 7.272 | 1.018 | 0.587 | 7.859 |
| 33 | SHTSGGSSNDNN | 6.418 | 6.272 | 1.825 | 1.506 | 7.778 |
| 34 | NETSGSSNDNA | 9.114 | 5.973 | 2.702 | 1.707 | 7.680 |
| 35 | STSAGGASNDNH | 6.525 | 5.769 | 8.894 | 1.865 | 7.634 |
| 36 | STTTGSSNDNN | 8.656 | 7.198 | 1.382 | 0.417 | 7.614 |
| 37 | SKSAGGSSNDNN | 6.200 | 4.994 | 3.161 | 2.533 | 7.527 |
| 38 | NHSTGATNDNA | 6.983 | 6.446 | 1.850 | 1.053 | 7.499 |
| 39 | NNSTGGATNDNA | 8.338 | 6.602 | 0.901 | 0.862 | 7.463 |
| 40 | STSAGGSTNDNP | 5.173 | 5.129 | 2.292 | 2.248 | 7.378 |
| 956 | SQSGASNDNH | 5.058 | 2.749 | −2.350 | −4.603 | −1.854 |

TABLE 11
Scores for AAVC11.11 VR-I variants-hFRG (no IVIg) replicate 2

| | | | Corrected | | Corrected | |
|---|---|---|---|---|---|---|
| | Insert | Entry | Entry | Expression | Expression | Total |
| Rank | sequence | Fitness | Fitness | Fitness | Fitness | Score |
| 1 | SKSGGGATNDNA | 11.881 | 10.271 | 6.656 | 5.740 | 16.012 |
| 2 | SKSDGASNDNA | 11.159 | 9.875 | 5.878 | 4.994 | 14.869 |
| 3 | NNTSGGSTNDNN | 16.020 | 9.664 | 16.854 | 4.830 | 14.494 |
| 4 | STSNGGSTNDNH | 10.643 | 9.990 | 4.647 | 3.811 | 13.801 |
| 5 | SDSSGGSTNDNH | 8.582 | 8.300 | 4.862 | 4.725 | 13.024 |
| 6 | NATAGGSSNDNH | 8.788 | 8.512 | 4.257 | 4.171 | 12.683 |
| 7 | SNTDGSSNDNT | 9.014 | 8.907 | 3.927 | 3.692 | 12.599 |
| 8 | NSTDGATNDNA | 8.907 | 8.791 | 4.220 | 3.407 | 12.198 |
| 9 | SESGSTNDNH | 9.494 | 8.996 | 3.133 | 3.021 | 12.017 |
| 10 | NQTSGATNDNA | 8.859 | 8.756 | 3.198 | 3.134 | 11.890 |
| 11 | NQTDGASNDNP | 11.245 | 9.309 | 4.115 | 2.571 | 11.880 |
| 12 | NKTSGASNDNA | 8.711 | 8.093 | 3.895 | 3.613 | 11.706 |
| 13 | SKTAGSSNDNA | 7.532 | 7.444 | 3.895 | 3.881 | 11.326 |
| 14 | SGTNGSSNDNA | 8.954 | 8.614 | 3.295 | 2.673 | 11.287 |
| 15 | NDSTGGATNDNN | 9.128 | 8.520 | 3.874 | 2.674 | 11.194 |
| 16 | NRTTGGATNDNP | 8.014 | 7.493 | 4.166 | 3.622 | 11.116 |
| 17 | SSTGGGSSNDNN | 7.505 | 7.155 | 4.224 | 3.939 | 11.095 |
| 18 | NTTTGATNDNT | 7.468 | 7.447 | 3.602 | 3.553 | 11.000 |
| 19 | SSSDGGATNDNT | 10.537 | 8.461 | 4.283 | 2.536 | 10.997 |
| 20 | NGSNGGSSNDNN | 7.017 | 6.958 | 3.888 | 3.839 | 10.798 |
| 21 | SQSAGSSNDNA | 8.608 | 8.412 | 2.814 | 2.342 | 10.754 |
| 22 | NQTAGGSSNDNH | 9.714 | 8.477 | 2.981 | 2.046 | 10.523 |
| 23 | SETGGGSSNDNP | 8.182 | 6.844 | 5.168 | 3.559 | 10.402 |
| 24 | STTDGSTNDNT | 7.457 | 6.906 | 3.606 | 3.453 | 10.359 |
| 25 | NQSSGGSSNDNH | 7.363 | 7.260 | 3.109 | 2.916 | 10.176 |
| 26 | SESNGGSTNDNH | 8.826 | 7.955 | 2.821 | 1.937 | 9.893 |
| 27 | SATNGSTNDNH | 8.621 | 8.413 | 1.736 | 1.450 | 9.864 |
| 28 | SESSGSSNDNA | 7.994 | 7.837 | 1.915 | 1.632 | 9.468 |
| 29 | SDTTGGSTNDNN | 8.487 | 8.133 | 2.626 | 1.226 | 9.358 |
| 30 | SESSGASNDNA | 7.710 | 7.673 | 1.715 | 1.597 | 9.270 |
| 31 | SQTDGGASNDNT | 7.754 | 7.538 | 2.242 | 1.611 | 9.149 |
| 32 | NQTDGASNDNA | 9.212 | 7.737 | 3.138 | 1.350 | 9.088 |
| 33 | NKSAGGATNDNT | 10.075 | 2.296 | 6.831 | 6.767 | 9.064 |
| 34 | SKSSGASNDNN | 8.936 | 7.858 | 2.465 | 1.135 | 8.993 |
| 35 | NSSSGSTNDNH | 7.350 | 6.546 | 3.059 | 2.248 | 8.793 |
| 36 | SKTTGASNDNA | 7.596 | 7.478 | 1.290 | 1.253 | 8.731 |
| 37 | STSTGASNDNT | 7.725 | 7.658 | 1.122 | 1.049 | 8.707 |
| 38 | NHSNGGASNDNN | 8.995 | 7.355 | 3.253 | 1.328 | 8.683 |
| 39 | SESSGGATNDNA | 7.639 | 7.623 | 0.976 | 0.886 | 8.509 |
| 40 | NNTTGGATNDNA | 8.479 | 5.076 | 4.599 | 3.376 | 8.452 |
| 624 | SQSGASNDNH | 1.709 | 1.067 | −0.271 | −2.487 | −1.420 |

TABLE 12
Scores for AAVC11.11 VR-I variants-hFRG + IVIg

| | | Entry |
|---|---|---|
| Rank | Insert sequence | Fitness |
| 1 | SKTTGGASNDNN | 8.915 |
| 2 | STTGGGATNDNP | 8.543 |
| 3 | STSGATNDNA | 7.232 |
| 4 | SKTSGSTNDNN | 7.189 |
| 5 | SATTGSTNDNP | 7.022 |
| 6 | SKTSGATNDNP | 6.924 |
| 7 | SNTTGGASNDNP | 6.452 |
| 8 | NKSAGGSTNDNH | 6.405 |
| 9 | NHSAGATNDNT | 6.297 |
| 10 | NRSDGSTNDNA | 6.280 |
| 11 | SDTDGGSSNDNP | 5.890 |
| 12 | SQSTGATNDNP | 5.762 |
| 13 | NGTAGGSSNDNN | 5.675 |
| 14 | SGTAGGSSNDNA | 5.647 |
| 15 | SGSNGGSTNDNT | 5.637 |
| 16 | NKSSGGATNDNH | 5.601 |
| 17 | SRTNGGSSNDNH | 5.353 |
| 18 | SSTTGASNDNP | 5.298 |
| 19 | SKTAGGASNDNA | 5.233 |
| 20 | SDSTGGSSNDND | 5.223 |
| 21 | NRSTGASNDNH | 5.199 |
| 22 | NASDGGSTNDNT | 5.159 |
| 23 | NETAGGSSNDND | 5.072 |
| 24 | NESGGATNDNH | 4.861 |
| 25 | NNTDGSSNDNN | 4.858 |
| 26 | NKSAGGATNDNH | 4.759 |
| 27 | NHSTGGSSNDNH | 4.714 |
| 28 | SSSTGGATNDNP | 4.706 |
| 29 | SQSGATNDNP | 4.701 |
| 30 | STTNGGSSNDNA | 4.609 |
| 31 | SRTGGGSSNDNA | 4.588 |
| 32 | SRSGSTNDNH | 4.559 |
| 33 | NQTTGGATNDNP | 4.540 |
| 34 | NPSGATNDNN | 4.523 |
| 35 | NRTNGGSTNDNP | 4.523 |
| 36 | SQSTGGSSNDNP | 4.486 |
| 37 | SSTGGSTNDNT | 4.431 |
| 38 | NASTGGSTNDNH | 4.385 |
| 39 | SSTSGASNDNH | 4.341 |
| 40 | SKTNGATNDNT | 4.304 |
| 64 | SQSGASNDNH | 3.868 |

TABLE 13
Scores for AAVC11.11 VR-I variants-liver explant

| | | | Corrected | | Corrected | |
|---|---|---|---|---|---|---|
| | | Entry | Entry | Expression | Expression | Total |
| Rank | Insert sequence | Fitness | Fitness | Fitness | Fitness | Score |
| 1 | NSSAGGATNDNP | 10.010 | 9.493 | 5.280 | 4.544 | 14.037 |
| 2 | NQSTGGATNDNP | 8.705 | 8.368 | 5.485 | 5.344 | 13.712 |
| 3 | NNSAGSSNDNP | 6.611 | 6.479 | 6.931 | 6.793 | 13.273 |
| 4 | SPTNGGSTNDNP | 8.691 | 8.662 | 4.602 | 4.452 | 13.114 |
| 5 | NASSGSTNDNA | 9.904 | 8.308 | 6.132 | 4.804 | 13.112 |
| 6 | NQSDGGSTNDNN | 8.827 | 8.657 | 4.132 | 4.108 | 12.765 |
| 7 | NQTSGGASNDNP | 8.777 | 8.680 | 2.798 | 2.583 | 11.263 |
| 8 | NASDGGSTNDNN | 7.623 | 7.623 | 4.877 | 3.278 | 10.902 |
| 9 | SDTAGATNDNP | 7.513 | 6.156 | 5.063 | 4.189 | 10.345 |
| 10 | SATTGSTNDNA | 7.661 | 7.230 | 3.980 | 2.822 | 10.051 |
| 11 | NNTGGATNDNA | 6.968 | 6.766 | 3.263 | 3.259 | 10.026 |
| 12 | NQTDGSTNDND | 7.377 | 7.318 | 2.485 | 2.470 | 9.787 |
| 13 | NNTGGATNDNP | 8.744 | 8.131 | 2.280 | 1.622 | 9.753 |
| 14 | SPTDGGSTNDNN | 9.623 | 8.408 | 4.737 | 1.316 | 9.724 |
| 15 | SETGGGSSNDNA | 5.952 | 4.674 | 7.087 | 4.485 | 9.158 |
| 16 | NPTSGGSTNDNN | 7.884 | 6.865 | 3.128 | 2.255 | 9.120 |
| 17 | SPSDGGSSNDNT | 6.451 | 6.325 | 2.678 | 2.677 | 9.002 |
| 18 | NETGSSNDNA | 9.289 | 8.956 | 3.280 | −0.173 | 8.782 |
| 19 | NTTSGSSNDNN | 8.460 | 8.217 | 0.422 | 0.373 | 8.591 |
| 20 | STTNGSTNDNA | 7.226 | 7.166 | 1.640 | 1.398 | 8.564 |
| 21 | NQSNGSTNDNN | 6.173 | 6.155 | 2.695 | 2.375 | 8.530 |
| 22 | NETGGSTNDNN | 9.744 | 7.534 | 4.263 | 0.949 | 8.483 |
| 23 | NKSAGGSSNDNH | 6.783 | 6.637 | 2.678 | 1.733 | 8.370 |
| 24 | NSSNGGASNDNP | 6.758 | 6.758 | 1.543 | 1.379 | 8.138 |
| 25 | NSSDGGATNDNT | 5.479 | 5.459 | 2.128 | 2.126 | 7.585 |
| 26 | NSTAGGATNDND | 5.932 | 5.824 | 1.555 | 1.544 | 7.368 |
| 27 | NQTAGSTNDNH | 5.787 | 5.774 | 1.488 | 1.459 | 7.233 |
| 28 | NQTTGGSTNDND | 6.009 | 4.880 | 2.395 | 2.304 | 7.184 |
| 29 | NNTAGGASNDNT | 8.165 | 8.134 | -0.846 | −1.022 | 7.112 |
| 30 | SHTGASNDNA | 7.412 | 7.402 | 1.585 | −0.448 | 6.954 |
| 31 | NASTGGATNDNT | 5.779 | 5.778 | 1.421 | 1.100 | 6.878 |
| 32 | NSSNGSTNDNH | 5.829 | 5.723 | 1.210 | 1.134 | 6.857 |
| 33 | NATTGGSTNDNN | 6.518 | 6.452 | 0.263 | 0.207 | 6.659 |
| 34 | NKSDGGSTNDNN | 5.731 | 5.704 | 0.847 | 0.838 | 6.542 |
| 35 | NDSTGASNDNT | 5.173 | 4.770 | 1.695 | 1.691 | 6.461 |
| 36 | NGTSGGSTNDND | 5.514 | 5.450 | 0.958 | 0.867 | 6.317 |
| 37 | NDTDGGSSNDNA | 6.367 | 5.221 | 1.637 | 1.038 | 6.259 |
| 38 | NQTTGSSNDNA | 5.446 | 5.188 | 1.903 | 1.010 | 6.198 |
| 39 | SQTGASNDNA | 5.887 | 5.393 | 0.836 | 0.761 | 6.154 |
| 40 | STSDGGSTNDNH | 7.344 | 7.183 | −0.739 | −1.054 | 6.130 |
| 2626 | SQSGASNDNH | 1.364 | 0.782 | −7.866 | −10.085 | −9.303 |

### Example 4. Generation of Secondary (2y) Libraries

Two Secondary (2y) libraries were constructed, one on the AAV-LK03-REDH capsid backbone and the other on the AAVC11.11 capsid backbone.

The AAV-LK03-REDH 2y library was constructed to contain the top 40 variants described in Tables 6-9 for the AAV-LK03-REDH VR-I library, with the exception of variant ‘STTSGASNDNA’, which was among the top 40 variants from the AAV-LK03-REDH VR-I library screened in an hFRG mouse (no IVIg) replicate 2 (Table 7) and among the top 40 variants from the AAV-LK03-REDH VR-I library screened in a hFRG mouse (with IVIg) (Table 8).

The AAVC11.11 2y library was constructed to contain the top 40 variants described in Tables 10-13 for the AAVC11.11 VR-I library. Accordingly, the AAV-LK03-REDH 2y library contained 159 selected variants and the AAVC11.11 2y library contained 160 selected variants.

Similar to the strategy followed with synonymous codons, each variant of the 2y library was ordered as two replicates (2 oligonucleotides per variant). To be able to use them as replicates, the first of the two oligonucleotides was constructed with the most frequently used codons in humans, and the second of the two oligonucleotides was constructed with the second most frequently used codons in humans. As an example, the sequence of the first oligonucleotide with the most frequently used codons for the wild-type VR-I (SQSGASNDNH; SEQ ID NO:12) is AGCCAGAGCGGCGCCAGCAACGACAACCAC (SEQ ID NO:341) and the sequence of the second oligonucleotide with the second most frequently used codons for the wild-type VR-I (SQSGASNDNH; SEQ ID NO:12) is TCCCAATCCGGAGCTTCCAATGATAATCAT (SEQ ID NO:342).

As both 2y libraries were synthesized as oligonucleotides, a set of controls were included for each library, corresponding to the wild-type VR-I sequence ‘SQSGASNDNH’ (SEQ ID NO:12), two negative VR-I controls (unselected variants) for each of the four previous selections (hFRG (no IVIg) replicate 1, hFRG (no IVIg) replicate 2, hFRG+IVIg, and liver explant), the VR-I amino acid region of wild-type AAV1, AAV7, AAV8, AAV9, and AAV10, and a variant harboring only stop codons. The synthesized oligonucleotides harbored also two homology regions upstream (5′-ACAACCATCTCTACAAGCAAATCTCC; SEQ ID NO:338) and downstream (5′-TACTTTGGCTACAGCACCCCTTGG; SEQ ID NO:339) of the VR-I region. Each 2y library was cloned into the respective background capsid with Gibson Assembly.

To distinguish between AAV-LK03-REDH and AAVC11.11 2y libraries that are jointly injected each library was further barcoded, as follows, at I240 (LK03-REDH numbering):

| | Amino Acid | Syn-Codon Capsid |
|---|---|---|
| REDH | ITT | ATCACAACA |
| AAVC11.11 | ITT | ATTACTACG |

Both 2y libraries were packaged independently, mixed at 1:1, and then injected into four different mouse models: 1) liver explant; 2) hFRG (no IVIg); 3) hFRG+IVIg as described in Example 3; and 4) PxB (no IVIg). Like FRG, PxB mice are also chimeric mice with a humanized liver. (PhoenixBio) DNA and cDNA were recovered from the FRG and PXB mice one month after injection and from the liver explant at days 2, 4, 6 and 8 days after injection.

The top ranked variants for the AAVC11.11 2y library from the liver explant model at the DNA (entry) level were determined based on the following criteria: 1) both synonymous codon replicates were among the top 100 variants; and 2) both synonymous codon replicates were present in at least 3 of the 4 days. The top variants based on their entry rank are provided in Table 14. Variants were designated LB11 2y 1-LB11 2y 8 (wildtype designated wt_LB11). These variants were also cloned into the AAVC11.12 capsid backbone (and were correspondingly designated LB12 2y 1-LB 12 2y 8).

TABLE 14
Top entry ranked variants for AAVC11.11 2y library-liver explant

| Selected VR-I region | SEQ ID NO | Designation¹ | Replicate 1 | Replicate 2 |
|---|---|---|---|---|
| SQSGASNDNH | 12 | wt_LB11 | 234 | 242 |
| SKTTGASNDNA | 253 | LB11 2y 1 | 1 | 5 |
| SKSSGASNDNN | 251 | LB11 2y 2 | 2 | 48 |
| NRSAGGSSNDNH | 196 | LB11 2y 3 | 3 | 82 |
| SRTNGGSSNDNH | 274 | LB11 2y 4 | 8 | 60 |
| NKSAGGSSNDNH | 319 | LB11 2y 5 | 12 | 55 |
| SRTGGGSSNDNA | 288 | LB11 2y 6 | 13 | 91 |
| NKSTGGATNDNT | 194 | LB11 2y 7 | 15 | 18 |
| NKTSGASNDNA | 231 | LB11 2y 8 | 17 | 44 |

¹Cloned into the AAVC11.12 backbone, designations are, correspondingly, LB12 2y 1-LB12 2y 8.

Expression cDNA reads from the liver explant model yielded only two top capsid variants. Equivalent ranks at the expression level are provided in Table 15. Variants were designated LB11 2y 9-LB11 2y 10. These variants were also cloned into the AAVC11.12 capsid backbone (and were correspondingly designated LB12 2y 9-LB 12 2y 10).

TABLE 15
Top expression ranked variants for AAVC11.11 2y library-liver explant

| Selected VR-I region | SEQ ID NO | Designation¹ | Replicate 1 | Replicate 2 |
|---|---|---|---|---|
| SKTGGATNDNA | 193 | LB11 2y 9 | 4 | 294 |
| NHSAGATNDNT | 266 | LB11 2y 10 | 241 | Not Found |

¹Cloned into the AAVC11.12 backbone, designations are, correspondingly, LB12 2y 9-LB12 2y 10.

The top variants for the AAVC11.11 2y library from the hFRG (no IVIg) (Table 16), hFRG+IVIg (Table 17) and PxB (no IVIg) (Table 18) models were also selected based on their entry and expression ranks. These variants were also cloned into the AAVC11.12 capsid backbone. Variants LB11 2y 11-LB11 2y 14 (Table 16) were selected based on their expression ranks. Variants LB11 2y 3, 7-9, and LB11 2y 15-17 (Table 17) were selected based on their expression ranks and variant LB11 2y 1, 12, and 18-20 (Table 17) were selected based on their entry ranks. Variants LB11 2y 21-LB11 2y 24 (Table 18) were selected based on their expression ranks and variant LB11 2y 25 (Table 18) was selected based on its entry rank.

TABLE 16
Top ranked variants for AAVC11.11 2y library-hFRG (no IVIg)

| Selected VR-I region | SEQ ID NO | Designation¹ | Entry Rank | Expression Rank |
|---|---|---|---|---|
| SKTTGGASNDNT | 207 | LB11 2y 11 | / | 2 |
| NKSSGGATNDNH | 273 | LB11 2y 12 | / | 5 |
| SESNGGSTNDNH | 243 | LB11 2y 13 | / | 6 |
| SQSGATNDNP | 286 | LB11 2y 14 | / | 7 |

¹Cloned into the AAVC11.12 backbone, designations are, correspondingly, LB12 2y 11-LB12 2y 14.

TABLE 17
Top ranked variants for AAVC11.11 2y library-hFRG + IVIg

| Selected VR-I region | SEQ ID NO | Designation¹ | Entry Rank | Expression Rank |
|---|---|---|---|---|
| NRSAGGSSNDNH | 196 | LB11 2y 3 | / | 2 |
| NKSTGGATNDNT | 194 | LB11 2y 7 | / | 3 |
| SSTNGGSSNDNH | 209 | LB11 2y 15 | / | 4 |
| SESSGGATNDNA | 256 | LB11 2y 16 | / | 5 |
| NKTSGASNDNA | 231 | LB11 2y 8 | / | 6 |
| NNTGGATNDNP | 310 | LB11 2y 17 | / | 9 |
| SKTGGATNDNA | 193 | LB11 2y 9 | / | 10 |
| SKTTGASNDNA | 253 | LB11 2y 1 | 1 | / |
| SKSGGGATNDNA | 220 | LB11 2y 18 | 2 | / |
| SKSTGSSNDNA | 198 | LB11 2y 19 | 3 | / |
| NKSSGGATNDNH | 273 | LB11 2y 12 | 4 | / |
| NKSAGGATNDNH | 283 | LB11 2y 20 | 5 | / |

¹Cloned into the AAVC11.12 backbone, designations are, correspondingly, LB12 2y 15-LB12 2y 20.

TABLE 18
Top ranked variants for AAVC11.11 2y library-PxB (no IVIg)

| Selected VR-I region | SEQ ID NO | Designation¹ | Entry Rank | Expression Rank |
|---|---|---|---|---|
| SSTTGASNDNP | 275 | LB11 2y 21 | / | 2 |
| STSNGGSTNDNH | 223 | LB11 2y 22 | / | 4 |
| SHTSGGSSNDNN | 212 | LB11 2y 23 | / | 5 |
| NSSNGSTNDNH | 328 | LB11 2y 24 | / | 9 |
| STSTGASNDNT | 254 | LB11 2y 25 | 2 | / |

¹Cloned into the AAVC11.12 backbone, designations are, correspondingly, LB12 2y 21-LB12 2y 25.

Some top variants for the AAV-LK03-REDH 2y library from the hFRG (no IVIg) (Table 19), hFRG+IVIg (Table 20) and PxB (no IVIg) (Table 21) models were also selected based on their entry and expression ranks. These variants were not cloned into the AAVC11.12 capsid backbone. Variants REDH 2y 1-REDH 2y 4 (Table 19) were selected based on their expression rank. Variants REDH 2y 5 and 6 (Table 20) were selected based on their expression rank. Variants REDH 2y 7-REDH 2y 9 (Table 21) were selected based on their expression ranks and variant REDH 2y 10 (Table 21) was selected based on its entry rank.

TABLE 19
Top ranked variants for AAV-LK03-REDH 2y library-hFRG (no IVIg)

| Selected VR-I region | SEQ ID NO | Designation | Entry Rank | Expression Rank |
|---|---|---|---|---|
| NRTGGGATNDNH | 67 | REDH 2y 1 | / | 1 |
| NPSAGGSSNDNN | 157 | REDH 2y 2 | / | 3 |
| NSSSGGATNDNT | 129 | REDH 2y 3 | / | 4 |
| NESGGGASNDNN | 64 | REDH 2y 4 | / | 8 |

TABLE 20
Top ranked variants for AAV-LK03-REDH 2y library-hFRG + IVIg

| Selected VR-I region | SEQ ID NO | Designation | Entry Rank | Expression Rank |
|---|---|---|---|---|
| NTTAGGSTNDNN | 117 | REDH 2y 5 | / | 1 |
| SKSSGATNDNA | 166 | REDH 2y 6 | / | 8 |

TABLE 21
Top ranked variants for AAV-LK03-REDH 2y library-PxB (no IVIg)

| Selected VR-I region | SEQ ID NO | Designation | Entry Rank | Expression Rank |
|---|---|---|---|---|
| NRTNGGATNDNT | 45 | REDH 2y 7 | / | 3 |
| SRSTGGASNDNA | 35 | REDH 2y 8 | / | 6 |
| NDSGGATNDNT | 164 | REDH 2y 9 | / | 7 |
| NKTGGASNDNT | 32 | REDH 2y 10 | 1 | / |

All of the selected/generated variants for the AAVC11.11 2y library and AAV-LK03-REDH 2y library, and those cloned onto the AAVC11.12 capsid backbone, totaling 60, were vectorized with two barcoded ITR2-LSP1-eGFP-Barcoded-WPRE-ITR2 transgenes. AAVC11.11, AAVC11.12, AAV-LK03 and AAV-LK03-REDH were used as controls.

The 60 selected/generated capsid variants were separated into two groups for validation in hFRG mice. Group 1 (34 variants) included the top ranked variants from the liver explant and hFRG+IVIg models for both AAVC11.11 2y and AAV-LK03-REDH 2y libraries, and the corresponding AAVC11.12 2y variants. Group 2 (26 variants) included the top ranked variants from the hFRG (no IVIg) and PxB models for both AAVC11.11 2y and AAV-LK03-REDH 2y libraries, and the corresponding AAVC11.12 2y variants.

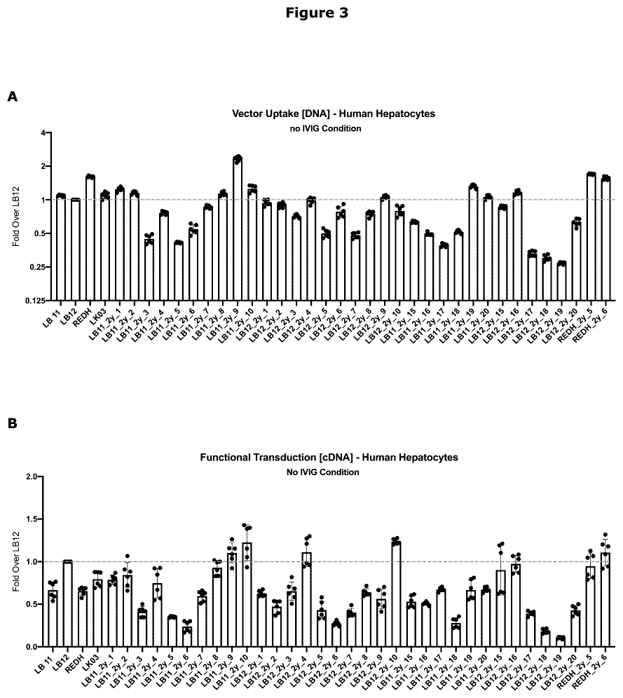

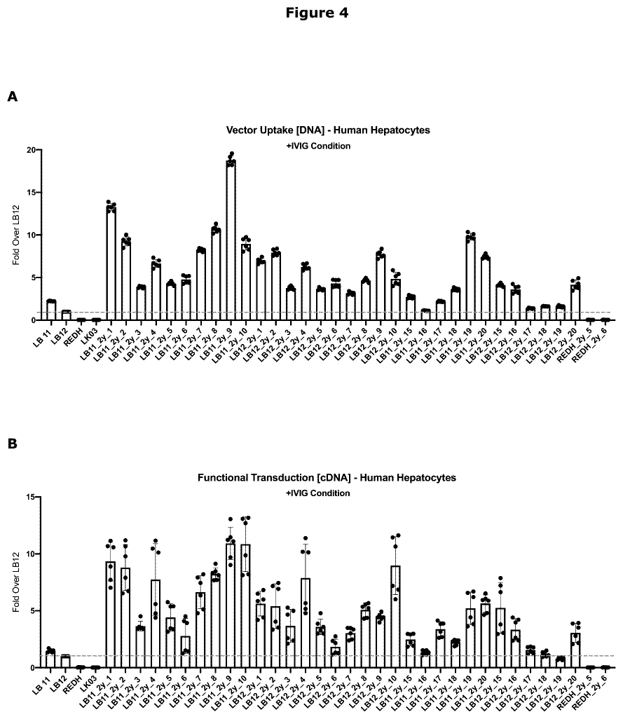

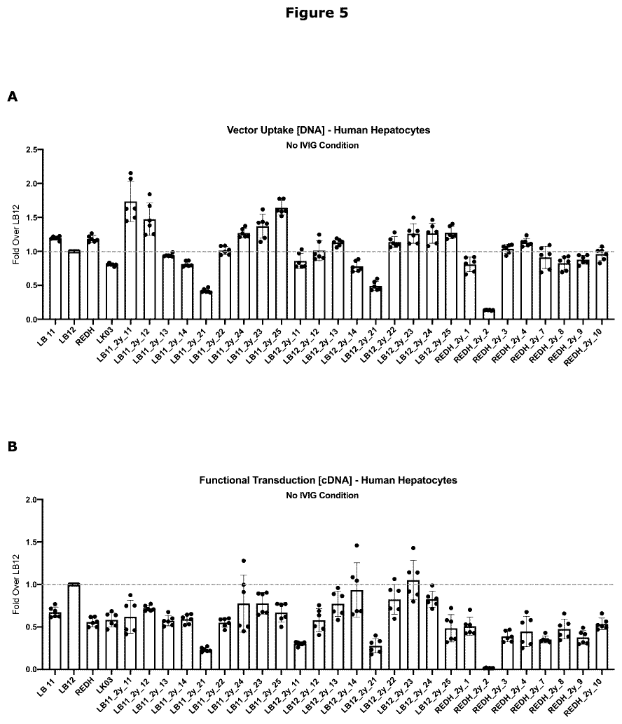

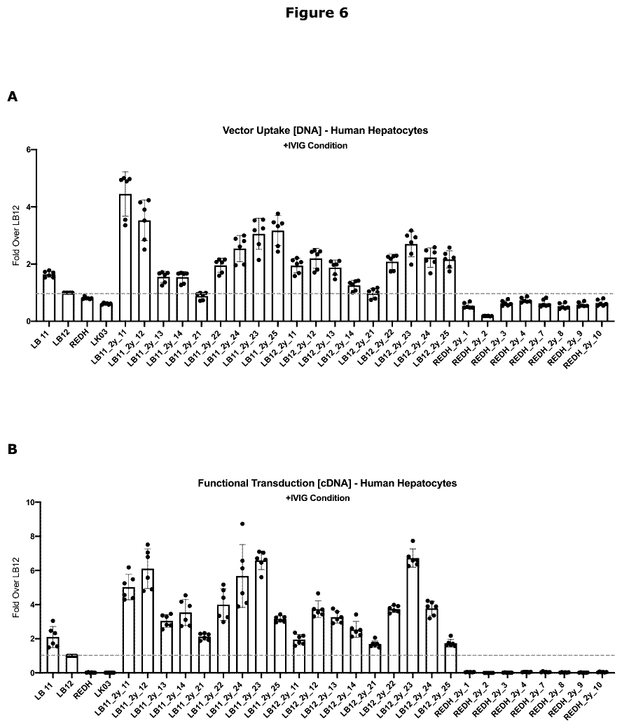

Each of the two selected/generated capsid variant groups were injected into either hFRG mice with or without IVIg (n=3 per group, total of 6 hFRG mice per capsid variant group) and compared against AAVC11.11, AAVC11.12, AAV-LK03 and AAV-LK03-REDH controls. Vector DNA and RNA (cDNA) levels in human hepatocytes isolated from the liver were assessed by NGS, with the data presented by normalizing to the pre-injection mix, and then normalized to the performance of the AAV-C11.12, by calculating as follows: (Capsid i DNA %/Capsid i Premix %)/(Capsid C11.C12 DNA %/Capsid C11.C12 Premix %). Results are shown in FIGS. 3 to 6. C11.C12 appears as 1, and the rest of the capsids indicate average fold change when compared to C11.C12 for the n=3 FRG per condition.

TABLE 22
Primer Sequences

| SEQ | | |
|---|---|---|
| ID NO | Name/description | Sequence (5′-3′) |
| 13 | Fwd 10 aa | CATCTCTACAAGCAAATCTCCARCVVWWCWGGAKCTWCWAACGACAACVMCTACTT |
| 14 | Fwd 11 aa | CATCTCTACAAGCAAATCTCCARCVVWWCWRVCGGAKCTWCWAACGACAACVMCTACTT |
| 15 | Fwd 12 aa | CATCTCTACAAGCAAATCTCCARCVVWWCWRVCGGAGGAKCTWCWAACGACAACVMCTA |
| | | CTT |
| 16 | Rev common 1 | GGAGATTTGCTTGTAGAGATGGTTGTTGTAAGTGGGCAGGGCCCAGGTTCTGG |
| 17 | Rev common 2 | GGAGATTTGCTTGTAGAGATGATTATTGTACGTGGGCAGGGCCCAGGTTCTGG |
| 18 | Rev common 3 | GGAGATTTGCTTGTAGAGATGGTTATTATAGGTGGGCAGGGCCCAGGTTCTGG |
| 19 | Forward NGS | ACCACCAGCACCAGAACCTGG |
| 20 | Reverse NGS | GCAGTGGAATCTGTTAAAGTCAAAATACCCCC |
| 337 | Forward NGS | ACTGAATGGCTGGGCGACAGAGTCAT |
| | (liver explant) | |
| 338 | 2y library | ACAACCATCTCTACAAGCAAATCTCC |
| | (upstream) | |
| 339 | 2y library | TACTTTGGCTACAGCACCCCTTGG |
| | (downstream) | |
| 341 | Codon-optimised | AGCCAGAGCGGCGCCAGCAACGACAACCAC |
| | 1 | |
| 342 | Codon-optimised | TCCCAATCCGGAGCTTCCAATGATAATCAT |
| | 2 | |

TABLE 23
Capsid Sequences

| SEQ | | |
|---|---|---|
| ID | | |
| NO | Name | Sequence |
| 1 | AAV-LK03- | ATGGCTGCTGACGGTTATCTTCCAGATTGGCTCGAGGACAACCTTTCTGAAGGCATTCG |
| | R594E + | AGAGTGGTGGGCGCTGCAACCTGGAGCCCCTAAACCCAAGGCAAATCAACAACATCAG |
| | D598H- | GACAACGCTCGGGGTCTTGTGCTTCCGGGTTACAAATACCTCGGACCCGGCAACGGACT |
| | VP1 (nt) | CGACAAGGGGGAACCCGTCAACGCAGCGGACGCGGCAGCCCTCGAGCACGACAAGGC |
| | | CTACGACCAGCAGCTCAAGGCCGGTGACAACCCCTACCTCAAGTACAACCACGCCGACG |
| | | CCGAGTTCCAGGAGCGGCTCAAAGAAGATACGTCTTTTGGGGGCAACCTCGGGCGAGC |
| | | AGTCTTCCAGGCCAAAAAGAGGCTTCTTGAACCTCTTGGTCTGGTTGAGGAAGCGGCTA |
| | | AGACGGCTCCTGGAAAGAAGAGGCCTGTAGATCAGTCTCCTCAGGAACCGGACTCATCA |
| | | TCTGGTGTTGGCAAATCGGGCAAACAGCCTGCCAGAAAAAGACTAAATTTCGGTCAGAC |
| | | TGGCGACTCAGAGTCAGTCCCAGACCCTCAACCTCTCGGAGAACCACCAGCAGCCCCCA |
| | | CAAGTTTGGGATCTAATACAATGGCTTCAGGCGGTGGCGCACCAATGGCAGACAATAAC |
| | | GAGGGTGCCGATGGAGTGGGTAATTCCTCAGGAAATTGGCATTGCGATTCCCAATGGCT |
| | | GGGCGACAGAGTCATCACCACCAGCACCAGAACCTGGGCCCTGCCCACTTACAACAACC |
| | | ATCTCTACAAGCAAATCTCCAGCCAATCAGGAGCTTCAAACGACAACCACTACTTTGGCT |
| | | ACAGCACCCCTTGGGGGTATTTTGACTTTAACAGATTCCACTGCCACTTCTCACCACGTG |
| | | ACTGGCAGCGACTCATTAACAACAACTGGGGATTCCGGCCCAAGAAACTCAGCTTCAAG |
| | | CTCTTCAACATCCAAGTTAAAGAGGTCACGCAGAACGATGGCACGACGACTATTGCCAA |
| | | TAACCTTACCAGCACGGTTCAAGTGTTTACGGACTCGGAGTATCAGCTCCCGTACGTGCT |
| | | CGGGTCGGCGCACCAAGGCTGTCTCCCGCCGTTTCCAGCGGACGTCTTCATGGTCCCTC |
| | | AGTATGGATACCTCACCCTGAACAACGGAAGTCAAGCGGTGGGACGCTCATCCTTTTAC |
| | | TGCCTGGAGTACTTCCCTTCGCAGATGCTAAGGACTGGAAATAACTTCCAATTCAGCTAT |
| | | ACCTTCGAGGATGTACCTTTTCACAGCAGCTACGCTCACAGCCAGAGTTTGGATCGCTTG |
| | | ATGAATCCTCTTATTGATCAGTATCTGTACTACCTGAACAGAACGCAAGGAACAACCTCT |
| | | GGAACAACCAACCAATCACGGCTGCTTTTTAGCCAGGCTGGGCCTCAGTCTATGTCTTTG |
| | | CAGGCCAGAAATTGGCTACCTGGGCCCTGCTACCGGCAACAGAGACTTTCAAAGACTGC |
| | | TAACGACAACAACAACAGTAACTTTCCTTGGACAGCGGCCAGCAAATATCATCTCAATGG |
| | | CCGCGACTCGCTGGTGAATCCAGGACCAGCTATGGCCAGTCACAAGGACGATGAAGAA |
| | | AAATTTTTCCCTATGCACGGCAATCTAATATTTGGCAAAGAAGGGACAACGGCAAGTAAC |
| | | GCAGAATTAGATAATGTAATGATTACGGATGAAGAAGAGATTCGTACCACCAATCCTGTG |
| | | GCAACAGAGCAGTATGGAACTGTGGCAAATAACTTGCAGAGCTCAAATACAGCTCCCAC |
| | | GACTGAAACTGTCAATCATCAGGGGGCCTTACCTGGCATGGTGTGGCAAGATCGTGACG |
| | | TGTACCTTCAAGGACCTATCTGGGCAAAGATTCCTCACACGGATGGACACTTTCATCCTT |
| | | CTCCTCTGATGGGAGGCTTTGGACTGAAACATCCGCCTCCTCAAATCATGATCAAAAATA |
| | | CTCCGGTACCGGCAAATCCTCCGACGACTTTCAGCCCGGCCAAGTTTGCTTCATTTATCA |
| | | CTCAGTACTCCACTGGACAGGTCAGCGTGGAAATTGAGTGGGAGCTACAGAAAGAAAAC |
| | | AGCAAACGTTGGAATCCAGAGATTCAGTACACTTCCAACTACAACAAGTCTGTTAATGTG |
| | | GACTTTACTGTAGACACTAATGGTGTTTATAGTGAACCTCGCCCCATTGGCACCCGTTAC |
| | | CTTACCCGTCCCCTGTAA |
| 2 | AAV-LK03- | MAADGYLPDWLEDNLSEGIREWWALQPGAPKPKANQQHQDNARGLVLPGYKYLGPGNGL |
| | R594E + | DKGEPVNAADAAALEHDKAYDQQLKAGDNPYLKYNHADAEFQERLKEDTSFGGNLGRAVF |
| | D598H- | QAKKRLLEPLGLVEEAAKTAPGKKRPVDQSPQEPDSSSGVGKSGKQPARKRLNFGQTGDS |
| | VP1 (aa) | ESVPDPQPLGEPPAAPTSLGSNTMASGGGAPMADNNEGADGVGNSSGNWHCDSQWLGD |
| | | RVITTSTRTWALPTYNNHLYKQISSQSGASNDNHYFGYSTPWGYFDFNRFHCHFSPRDWQ |
| | | RLINNNWGFRPKKLSFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFQFSYTFEDVPF |
| | | HSSYAHSQSLDRLMNPLIDQYLYYLNRTQGTTSGTTNQSRLLFSQAGPQSMSLQARNWLP |
| | | GPCYRQQRLSKTANDNNNSNFPWTAASKYHLNGRDSLVNPGPAMASHKDDEEKFFPMHG |
| | | NLIFGKEGTTASNAELDNVMITDEEEIRTTNPVATEQYGTVANNLQSSNTAPTTETVNHQGA |
| | | LPGMVWQDRDVYLQGPIWAKIPHTDGHFHPSPLMGGFGLKHPPPQIMIKNTPVPANPPTTF |
| | | SPAKFASFITQYSTGQVSVEIEWELQKENSKRWNPEIQYTSNYNKSVNVDFTVDTNGVYSE |
| | | PRPIGTRYLTRPL |
| 3 | AAVC11.11 | ATGGCTGCCGATGGTTATCTTCCAGATTGGCTCGAGGACACTCTCTCTGAAGGCATTCG |
| | (nt) | CGAGTGGTGGGCGCTGAAACCTGGAGCTCCACAACCCAAGGCCAACCAACAGCATCAG |
| | | GACAACGGCAGGGGTCTTGTGCTTCCTGGGTACAAGTACCTCGGACCCTTCAACGGACT |
| | | CGACAAGGGAGAGCCGGTCAACGAGGCAGACGCCGCGGCCCTCGAGCACGACAAGGC |
| | | CTACGACAAGCAGCTCGAGCAGGGGGACAACCCGTACCTCAAGTACAACCACGCCGAC |
| | | GCCGAGTTTCAGGAGCGTCTGCAAGAAGATACGTCTTTTGGGGGCAACCTCGGGCGAG |
| | | CAGTCTTCCAGGCCAAGAAGCGGGTTCTCGAACCTCTCGGTCTGGTTGAGGAAGGCGCT |
| | | AAGACGGCTCCTGGAAAGAAGAGACCGGTAGAGCCGTCACCTCAGCGTTCCCCCGACT |
| | | CCTCCACGGGCATCGGCAAGAAAGGCCAGCAGCCCGCCAGAAAGAGACTCAATTTCGG |
| | | TCAGACTGGCGACTCAGAGTCAGTCCCCGACCCTCAACCTCTCGGAGAACCTCCAGCAG |
| | | CGCCCTCTAGTGTGGGATCTGGTACAGTGGCTGCAGGCGGTGGCGCACCAATGGCAGA |
| | | CAATAACGAAGGTGCCGACGGAGTGGGTAATGCCTCAGGAAATTGGCATTGCGATTCCA |
| | | CATGGCTGGGCGACAGAGTCATTACCACCAGCACCCGAACCTGGGCCCTGCCCACCTAC |
| | | AACAACCACCTCTACAAGCAAATCTCCAGCCAATCAGGAGCTTCAAACGACAACCACTAC |
| | | TTTGGCTACAGCACCCCTTGGGGGTATTTTGACTTTAACAGATTCCACTGCCACTTCTCA |
| | | CCACGTGACTGGCAGCGACTCATTAACAACAACTGGGGATTCCGGCCCAAGAGACTCAA |
| | | CTTCAAGCTCTTCAACATCCAAGTCAAGGAGGTCACGACGAATGATGGCGTCACGACCA |
| | | TCGCTAATAACCTTACCAGCACGGTTCAAGTCTTCTCGGACTCGGAGTACCAGTTGCCGT |
| | | ACGTCCTCGGCTCTGCGCACCAGGGCTGCCTCCCTCCGTTCCCGGCGGACGTGTTCATG |
| | | ATTCCCCAGTACGGCTACCTAACACTCAACAACGGTAGTCAGGCCGTGGGACGCTCCTC |
| | | CTTTTACTGCCTGGAATATTTCCCATCGCAGATGCTGAGAACGGGCAATAACTTTGAGTT |
| | | CAGCTACAGCTTCGAGGACGTGCCTTTCCACAGCAGCTACGCACACAGCCAGAGCTTGG |
| | | ACCGACTGATGAATCCTCTCATTGACCAGTACCTGTACTACTTATCCAGAACTCAGTCCA |
| | | CAGGAGGAACTCAAGGTACCCAGCAATTGTTATTTTCTCAAGCTGGGCCTGCAAACATG |
| | | TCGGCTCAGGCCAAGAACTGGCTGCCTGGACCTTGCTACCGGCAGCAGCGAGTCTCCA |
| | | CGACACTGTCGCAAAACAACAACAGCAACTTTGCTTGGACTGGTGCCACCAAATATCACC |
| | | TGAACGGCAGAAACTCGTTGGTTAATCCCGGCGTCGCCATGGCAACTCACAAGGACGAC |
| | | GAGGACCGCTTTTTCCCATCCAGCGGAGTCCTGATTTTTGGAAAAACTGGAGCAACTAA |
| | | CAAAACTACATTGGAAAATGTGTTAATGACAAATGAAGAAGAAATTCGTCCTACTAATCC |
| | | TGTAGCCACGGAAGAATACGGGATAGTCAGCAGCAACTTACAAGCGGCTAATACTGCAG |
| | | CCCAGACACAAGTTGTCAACAACCAGGGAGCCTTACCTGGCATGGTCTGGCAGAACCGG |
| | | GACGTGTACCTGCAGGGTCCCATTTGGGCCAAAATTCCTCACACAGATGGACACTTTCA |
| | | CCCGTCTCCTCTTATGGGCGGCTTTGGACTCAAGAACCCGCCTCCTCAGATCCTCATCAA |
| | | AAACACGCCTGTTCCTGCGAATCCTCCGGCGGAGTTTTCAGCTACAAAGTTTGCTTCATT |
| | | CATCACCCAGTATTCCACAGGACAAGTGAGCGTGGAGATTGAATGGGAGCTGCAGAAA |
| | | GAAAACAGCAAACGCTGGAATCCCGAAGTGCAGTATACATCTAACTATGCAAAATCTGC |
| | | CAACGTTGATTTCACTGTGGACAACAATGGACTTTATACTGAGCCTCGCCCCATTGGCAC |
| | | CCGTTACCTTACCCGTCCCCTGTAA |
| 4 | AAVC11.11 | MAADGYLPDWLEDTLSEGIREWWALKPGAPQPKANQQHQDNGRGLVLPGYKYLGPFNGL |
| | (aa) | DKGEPVNEADAAALEHDKAYDKQLEQGDNPYLKYNHADAEFQERLQEDTSFGGNLGRAVF |
| | | QAKKRVLEPLGLVEEGAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYSFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQAANTAAQTQVNNQGALPG |
| | | MVWQNRDVYLQGPIWAKIPHTDGHFHPSPLMGGFGLKNPPPQILIKNTPVPANPPAEFSAT |
| | | KFASFITQYSTGQVSVEIEWELQKENSKRWNPEVQYTSNYAKSANVDFTVDNNGLYTEPRP |
| | | IGTRYLTRPL |
| 5 | AAV2 | MAADGYLPDWLEDTLSEGIRQWWKLKPGPPPPKPAERHKDDSRGLVLPGYKYLGPFNGLD |
| | prototypic | KGEPVNEADAAALEHDKAYDRQLDSGDNPYLKYNHADAEFQERLKEDTSFGGNLGRAVFQ |
| | capsid-VP1 | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVPANPSTTFSAA |
| | | KFASFITQYSTGQVSVEIEWELQKENSKRWNPEIQYTSNYNKSVNVDFTVDTNGVYSEPRP |
| | | IGTRYLTRNL |
| 6 | AAV1 | ALPTYNNHLYKQISSASTGASNDNHYFGYSTPWG |
| | capsid | |
| | positions | |
| | 248-281 | |
| 7 | AAV2/3b | ALPTYNNHLYKQISSQSGASNDNHYFGYSTPWG |
| | capsid | |
| | positions | |
| | 248-280 | |
| 8 | AAV7 | ALPTYNNHLYKQISSETAGSTNDNTYFGYSTPWG |
| | capsid | |
| | positions | |
| | 249-282 | |
| 9 | AAV8 | ALPTYNNHLYKQISSGTSGGATNDNTYFGYSTPWG |
| | capsid | |
| | positions | |
| | 249-283 | |
| 10 | AAV9 | ALPTYNNHLYKQISNSTSGGSSNDNAYFGYSTPWG |
| | capsid | |
| | positions | |
| | 248-282 | |
| 11 | AAV10 | ALPTYNNHLYKQISNGTSGGSTNDNTYFGYSTPWG |
| | capsid | |
| | positions | |
| | 249-283 | |
| 12 | AAV2 | SQSGASNDNH |
| | capsid | |
| | positions | |
| | 262-271 | |
| 340 | AAVC11.12 | MAADGYLPDWLEDTLSEGIREWWALKPGAPQPKANQQHQDNGRGLVLPGYKYLGPFNGL |
| | (aa) | DKGEPVNEADAAALEHDKAYDKQLEQGDNPYLKYNHADAEFQERLQEDTSFGGNLGRAVF |
| | | QAKKRILEPLGLVEEAAKTAPGKKRPVEPSPQRSPDSSTGIGKKGQQPARKRLNFGQTGDS |
| | | ESVPDPQPLGEPPAAPSSVGSGTVAAGGGAPMADNNEGADGVGNASGNWHCDSTWLGD |
| | | RVITTSTRTWALPTYNNHLYKQISSQSGASNDNHYFGYSTPWGYFDFNRFHCHFSPRDWQ |
| | | RLINNNWGFRPKRLSFKLFNIQVKEVTTNDGVTTIANNLTSTVQVFSDSEYQLPYVLGSAHQ |
| | | GCLPPFPADVFMIPQYGYLTLNNGSQAVGRSSFYCLEYFPSQMLRTGNNFEFSYTFEDVPFH |
| | | SSYAHSQSLDRLMNPLIDQYLYYLSRTQSTGGTQGTQQLLFSQAGPANMSAQAKNWLPGP |
| | | CYRQQRVSTTLSQNNNSNFAWTGATKYHLNGRNSLVNPGVAMATHKDDEDRFFPSSGVLI |
| | | FGKTGATNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPPEVFTPA |
| | | KFASFITQYSTGQVSVEIEWELQKENSKRWNPEIQYTSNYNKSVSVDFTVDTNGVYSEPRP |
| | | IGTRYLTRNL |

1. A capsid polypeptide comprising a variant VR-I, wherein:
the variant VR-I comprises a sequence set forth in any one of SEQ ID NOs: 21-336; and
the capsid polypeptide comprises a sequence having at least or about 85%, 86%, 87%, 88%, 89%, 90%, 91%, 92%, 93%, 94%, 95%, 96%, 97%, 98% or 99% sequence identity to: (i) the sequence set forth in SEQ ID NO:2; (ii) the sequence of amino acids at positions 138-735 of SEQ ID NO:2; or (iii) the sequence of amino acids at positions 203-736 of SEQ ID NO:2.

2-23. (canceled)
